(19) United States
(12) Patent Application Publication
(10) Pub. No.: US 2024/0141418 A1
(43) Pub. Date: May 2, 2024

(54) METHODS, COMPOSITIONS, AND SYSTEMS FOR SPATIAL ANALYSIS OF BIOLOGICAL SAMPLES

(71) Applicant: 10x Genomics, Inc., Pleasanton, CA (US)

(72) Inventor: Paulius MIELINIS, Hagersten (SE)

(21) Appl. No.: 18/496,492

(22) Filed: Oct. 27, 2023

Related U.S. Application Data
(60) Provisional application No. 63/420,436, filed on Oct. 28, 2022.

(51) Int. Cl. C12Q 1/6844; C12Q 1/6841

(52) U.S. Cl. CPC C12Q 1/6844; C12Q 1/6841

(57) ABSTRACT

The present disclosure relates in some aspects to methods, compositions, and kits for profiling biological targets in a sample using rolling circle amplification of circularized probes captured on a substrate.

### CROSS-REFERENCE TO RELATED APPLICATION

This application claims priority to U.S. Provisional Patent Application No. 63/420,436, filed Oct. 28, 2022, entitled “Methods, Compositions, and Systems for Improved Spatial Analysis of Biological Samples,” which is herein incorporated by reference in its entirety for all purposes.

### FIELD

The present disclosure relates in some aspects to methods, compositions, and systems for spatial analysis of biological samples by rolling circle amplification.

### BACKGROUND

Profiling biological targets in a sample, such as genomic, transcriptomic, or proteomic profiling of cells, are essential for many purposes, such as understanding the molecular basis of cell identity and developing treatment for diseases. Microscopy imaging, which can resolve multiple analytes in a sample, provides valuable information such as analyte abundance and spatial information of analytes in situ. Current in situ hybridization and sequencing-based approaches suffer from low efficiency, but the potential value of such in-tissue analysis could be enormous. Therefore, there is a need for new and improved methods for analyzing analytes and their relative spatial locations in a biological sample.

### SUMMARY

In some aspects, the present application provides new and improved methods, compositions, and kits for profiling biological targets (analytes) in a sample using rolling circle amplification. In some aspects, circularizable probes or probe sets are circularized using target nucleic acid sequences that are in or are associated with analytes in the biological sample as templates for circularization.

Unlike existing “in situ” rolling circle amplification assays wherein rolling circle amplification is performed in the biological sample and the sample is then imaged to detect the rolling circle amplification products at their locations in the sample, the provided methods comprise capturing the circularized probes using immobilized oligonucleotide molecules on a substrate that is aligned with the biological sample, and performing rolling circle amplification using the immobilized oligonucleotide molecules as primers. The rolling circle amplification products are then detected at locations on the substrate. In some aspects, the disclosed methods, compositions, kits, and systems facilitate rolling circle amplification (RCA) and detection of rolling circle amplification products (RCPs) on a substrate rather than in a biological sample, allowing for more efficient reactions and washes and reducing background fluorescence. In some aspects, data obtained from spatial analysis of the RCPs on the substrate is superimposed with data obtained from imaging the biological sample to correlate the locations of the detected RCPs with locations in the biological sample (e.g., tissue morphology or other features of the sample).

In some aspects, provided herein is a method of analyzing a biological sample, comprising: a) contacting the biological sample with a circularizable probe or probe set that hybridizes to a target nucleic acid sequence in the biological sample; b) ligating the circularizable probe or probe set to generate a circularized probe at a location in the biological sample; c) transferring the circularized probe from the biological sample to a substrate comprising a plurality of oligonucleotide molecules immobilized on the substrate, wherein at least a portion of the immobilized oligonucleotide molecules comprise a capture region complementary to a primer binding sequence of the circularized probe; d) performing rolling circle amplification of the circularized probe using the capture region as a primer, thereby generating a rolling circle amplification product immobilized at a location on the substrate; and e) detecting the rolling circle amplification product at the location on the substrate. In some embodiments, the capture region comprises a primer sequence.

In some aspects, provided herein is a method of analyzing a biological sample, comprising: a) contacting the biological sample with a circularizable probe or probe set that binds to a target nucleic acid sequence in the biological sample; b) ligating the circularizable probe or probe set to generate a circularized probe at a location in the biological sample, wherein the circularized probe comprises a primer binding sequence; c) transferring the circularized probe from the biological sample to a substrate comprising a plurality of oligonucleotide molecules immobilized on the substrate, wherein at least a portion of the immobilized oligonucleotide molecules comprise a primer sequence, and wherein the primer sequence is bound to the primer binding sequence of the circularized probe; d) using a polymerase which is optionally a Phi29 polymerase and the primer sequence to perform rolling circle amplification of the circularized probe, thereby generating a rolling circle amplification product immobilized at a location on the substrate; and e) detecting the rolling circle amplification product at the location on the substrate.

In some embodiments, contacting the biological sample with the circularizable probe or probe set in a) comprises contacting the biological sample with a pool of circularizable probes or probe sets that hybridize to a plurality of different target nucleic acid sequences in the biological sample, wherein the pool comprises the circularizable probe or probe set, and wherein: the ligating in b) comprises ligating at least a subset of the plurality of circularizable probes or probe sets to generate a plurality of circularized probes at two or more locations in the biological sample; the transferring in c) comprises transferring at least a subset of the circularized probes from the biological sample to the substrate; performing rolling circle amplification in d) comprises performing rolling circle amplification of at least a subset of the circularized probes using the capture region as a primer, thereby generating a plurality of rolling circle amplification products immobilized at two or more locations on the substrate; and the detecting in e) comprises detecting at least a subset of the rolling circle amplification products at the two or more locations on the substrate.

In some embodiments, contacting the biological sample with the circularizable probe or probe set in a) comprises contacting the biological sample with a pool of circularizable probes or probe sets that bind to a plurality of different target nucleic acid sequences in the biological sample, wherein the pool comprises the circularizable probe or probe set, and wherein: the ligating in b) comprises ligating at least a subset of the plurality of circularizable probes or probe sets to generate a plurality of circularized probes at two or more locations in the biological sample; the transferring in c) comprises transferring at least a subset of the circularized probes from the biological sample to the substrate; performing rolling circle amplification in d) comprises performing rolling circle amplification of at least a subset of the circularized probes using the polymerase and the primer sequence, thereby generating a plurality of rolling circle amplification products immobilized at two or more locations on the substrate; and the detecting in e) comprises detecting at least a subset of the rolling circle amplification products at the two or more locations on the substrate.

In some aspects, provided herein is a method of analyzing a biological sample, comprising: a) contacting the biological sample with a plurality of circularizable probes or probe sets that hybridize to a plurality of target nucleic acid sequences in the biological sample; b) ligating the plurality of circularizable probes or probe sets to generate a plurality of circularized probes; c) transferring the plurality of circularized probes from the biological sample to a substrate comprising a plurality of oligonucleotide molecules immobilized on the substrate, wherein at least a portion of the plurality of oligonucleotide molecules comprise a capture region that binds to the plurality of circularized probes; d) amplifying the plurality of circularized probe using rolling circle amplification using the capture region as a primer, thereby generating a plurality of rolling circle amplification products immobilized at a plurality of locations on the substrate; and e) detecting the plurality of rolling circle amplification products at the plurality of locations on the substrate.

In some aspects, provided herein is a method of analyzing a biological sample, comprising: a) contacting the biological sample with a plurality of circularizable probes or probe sets that bind to a plurality of target nucleic acid sequences in the biological sample; b) ligating the plurality of circularizable probes or probe sets to generate a plurality of circularized probes; c) transferring the plurality of circularized probes from the biological sample to a substrate comprising a plurality of oligonucleotide molecules immobilized on the substrate, wherein at least a portion of the plurality of oligonucleotide molecules comprise a primer sequence that binds to the plurality of circularized probes; d) using a polymerase which is optionally a Phi29 polymerase and the primer sequence to amplify the plurality of circularized probes by rolling circle amplification, thereby generating a plurality of rolling circle amplification products immobilized at a plurality of locations on the substrate; and e) detecting the plurality of rolling circle amplification products at the plurality of locations on the substrate.

In any of the preceding embodiments, the biological sample can be on a first substrate in the contacting in a), and the substrate comprising the plurality of oligonucleotide molecules immobilized on the substrate is a second substrate. In some embodiments, the biological sample may not come into direct contact with the second substrate.

In any of the preceding embodiments, the immobilized oligonucleotide molecules may be uniformly distributed on a surface of the substrate. In any of the preceding embodiments, the immobilized oligonucleotide molecules may be distributed over a capture area on the substrate, wherein the capture area corresponds to an area aligned with the biological sample. In any of the preceding embodiments, the immobilized oligonucleotide molecules may be uniformly distributed over a capture area on the substrate, wherein the capture area corresponds to an area aligned with the biological sample. In some embodiments, the immobilized oligonucleotide molecules are uniformly distributed over the capture area. In any of the preceding embodiments, the immobilized oligonucleotide molecules may cover at least 60%, 70%, 80%, 90%, or 95% of the capture area. In any of the preceding embodiments, the immobilized oligonucleotide molecules may cover at least 95%, 96%, 97%, 98%, or 99% of the capture area. In some embodiments, the immobilized oligonucleotide molecules cover 100% of the capture area. In any of the preceding embodiments, the immobilized oligonucleotide molecules may but do not need to be distributed in a pattern of discrete features on the capture area. In some embodiments, the substrate is prepared by dipping a functionalized substrate (e.g., an NETS-coated substrate, such as a slide) in an oligonucleotide solution to form a lawn of immobilized on the substrate.

In any of the preceding embodiments, the capture region can be a common sequence among a plurality of the oligonucleotide molecules. In some cases, the capture region can be a specific sequence to capture a corresponding circularized probe. In some cases, a subset of the plurality of oligonucleotide molecules can share the same sequence in the capture region (e.g. specific for a subset of the circularized probes). In any of the preceding embodiments, the immobilized oligonucleotide molecules can be at least 25 nucleotides in length. In any of the preceding embodiments, the immobilized oligonucleotide molecules can be at least 50, 60, 70, or 75 or more nucleotides in length. In any of the preceding embodiments, the capture region may be between about 10 and about 25 nucleotides in length. In any of the preceding embodiments, the capture region may be at the 3′ end of the immobilized oligonucleotide molecules. In any of the preceding embodiments, the melting temperature (Tₘ) of the capture region and the primer binding sequence may be between about 30° C. and about 90° C. In any of the preceding embodiments, the melting temperature (Tₘ) of the capture region and the primer binding sequence may be between about 40° C. and about 70° C.

In any of the preceding embodiments, the primer sequence can be a common sequence among at least two of the immobilized oligonucleotide molecules. In some cases, the primer sequence can be a specific sequence to capture a corresponding circularized probe. In some cases, a subset of the plurality of oligonucleotide molecules can share the same sequence in the primer sequence (e.g. specific for a subset of the circularized probes). In any of the preceding embodiments, the immobilized oligonucleotide molecules can be at least 25 nucleotides in length. In any of the preceding embodiments, the immobilized oligonucleotide molecules can be at least 50, 60, 70, or 75 or more nucleotides in length. In any of the preceding embodiments, the primer sequence may be between about 10 and about 25 nucleotides in length. In any of the preceding embodiments, the primer sequence may be at the 3′ end of the immobilized oligonucleotide molecules. In any of the preceding embodiments, the melting temperature (Tₘ) of the primer sequence and the primer binding sequence may be between about 30° C. and about 90° C. In any of the preceding embodiments, the melting temperature (Tₘ) of the primer sequence and the primer binding sequence may be between about 40° C. and about 70° C.

In any of the preceding embodiments, the capture region can be connected to the substrate via a linker. In some embodiments, the capture region can be connected to the substrate via a flexible linker. In any of the preceding embodiments, the flexible linker can be a nucleic acid sequence between the capture region and an immobilized end of the immobilized oligonucleotide molecules. In any of the preceding embodiments, the linker can be a nucleic acid sequence of between about 5 and about 100 nucleotides in length between the capture region and an immobilized end of the immobilized oligonucleotide molecules. Alternatively or additionally, the linker can be an organic linker.

In any of the preceding embodiments, the primer sequence can be connected to the substrate via a linker. In some embodiments, the primer sequence can be connected to the substrate via a flexible linker. In any of the preceding embodiments, the flexible linker can be a nucleic acid sequence between the primer sequence and an immobilized end of the immobilized oligonucleotide molecules. In any of the preceding embodiments, the flexible linker can be a nucleic acid sequence of between about 5 and about 100 nucleotides in length between the primer sequence and an immobilized end of the immobilized oligonucleotide molecules. Alternatively or additionally, the linker can be an organic linker.

In any of the preceding embodiments, the immobilized oligonucleotide molecules can be immobilized on the substrate via their 5′ ends. In any of the preceding embodiments, the immobilized oligonucleotide molecules can comprise 3′ ends capable of being extended by the polymerase. In any of the preceding embodiments, the immobilized oligonucleotide molecules can be immobilized on the substrate via their 5′ ends and comprise 3′ ends capable of being extended by the polymerase. In any of the preceding embodiments, the immobilized oligonucleotide molecules may comprise free 3′ hydroxyl groups. In any of the preceding embodiments, the immobilized oligonucleotide molecules can be immobilized on the substrate via a 5′ amino group.

In any of the preceding embodiments, the biological sample can be a cell or tissue sample, optionally wherein the biological sample is a tissue section. In some embodiments, the biological sample is a fresh tissue section. In some embodiments, the biological sample is a fresh frozen tissue section. In some embodiments, the biological sample is a paraffin embedded formalin fixed (FFPE) tissue section. In some embodiments, the method further comprises de-crosslinking the biological sample before contacting the biological sample with the circularizable probe or probe set or the pool of circularizable probes or probe sets. In some embodiments, the de-crosslinking comprises incubating the biological sample in a citrate buffer. In some embodiments, an FFPE tissue sample is pre-permeabilized with a proteinase (e.g., proteinase K).

In any of the preceding embodiments, the target nucleic acid sequence can be DNA. In any of the preceding embodiments, the target nucleic acid sequence can be RNA. In any of the preceding embodiments, the target nucleic acid sequence can be in an endogenous nucleic acid analyte in the biological sample. In any of the preceding embodiments, the target nucleic acid sequence can be in or associated with a labeling agent associated with a nucleic acid or non-nucleic acid analyte in the biological sample.

In any of the preceding embodiments, the method can comprise releasing the circularized probe from the target nucleic acid sequence. In some embodiments, the circularized probe is released from the target nucleic acid sequence using an endonuclease. In some embodiments, the endonuclease is an RNase. In some embodiments, the RNase is RNase H.

In any of the preceding embodiments, the biological sample can be permeabilized prior to transferring the circularized probe or the plurality of circularized probes to the substrate. In some embodiments, the method may comprise contacting the biological sample with proteinase K and/or RNase H to permeabilize the biological sample.

In any of the preceding embodiments, the method can comprise imaging the biological sample. In some embodiments, the method comprises staining the biological sample prior to imaging the biological sample. In some embodiments, the biological sample is stained with a nuclear stain, a histological stain, and/or an immunologic stain.

In any of the preceding embodiments, the method can comprise correlating the location or locations of the rolling circle amplification product(s) on the substrate with a location or locations in an image of the biological sample. In any of the preceding embodiments, the substrate can comprise a plurality of fiducial marks. In some embodiments, the fiducial marks are used to help correlate the location or locations of the rolling circle amplification product(s) on the substrate with the location or locations in the image of the biological sample. In any of the preceding embodiments, the fiducial marks may be fluorescent.

In any of the preceding embodiments, the rolling circle amplification may be performed in a buffer comprising a crowding agent. In some embodiments, the crowding agent is selected from the group consisting of poly(ethylene glycol) (PEG), glycerol, Ficoll®, and dextran sulfate. In some embodiments, the crowding agent is poly(ethylene glycol) (PEG). In some embodiments, the PEG is selected from the group consisting of PEG200, PEG8000, and PEG35000. In any of the preceding embodiments, the buffer may comprise between about 5% and about 15% PEG. In some embodiments, the buffer comprises about 10% PEG. In some embodiments, the crowding agent is and dextran sulfate.

In any of the preceding embodiments, the rolling circle amplification may be performed using a Phi29 polymerase. In any of the preceding embodiments, the rolling circle amplification may be performed for no more than 60, 90, or 120 minutes. In any of the preceding embodiments, the diameters of at least 90% of the rolling circle amplification products (RCP) may be within 3 μm of the median RCP diameter.

In any of the preceding embodiments, the method can comprise performing one or more wash step after ligating the circularizable probe(s) or probe set(s). In some embodiments, the one or more wash step is performed using phosphate buffered saline comprising a non-ionic surfactant.

In any of the preceding embodiments, the detecting can comprise detecting a signal or signals corresponding to the rolling circle amplification product or products. In some embodiments, detecting the signal or signals comprises detecting a detectably labeled probe or probes bound directly or indirectly to the rolling circle amplification product or products. In some embodiments, the detectably labeled probe binds to an intermediate probe that binds to the rolling circle amplification product. In some embodiments, the intermediate probe comprises: (i) a recognition sequence complementary to a barcode sequence or portion thereof in the rolling circle amplification product or products, and (ii) a binding site for the detectably labeled probe or probes. In any of the preceding embodiments, the detecting can comprise sequential binding of probes to the rolling circle amplification product(s) and detection of the probes bound to the rolling circle amplification product(s). In any of the preceding embodiments, the detecting may comprise determining a sequence of the rolling circle amplification product or products using sequencing-by-ligation (SBL) or base-by-base sequencing (e.g., sequencing-by-synthesis (SBS)). In any of the preceding embodiments, the immobilized oligonucleotide molecules may but do not need to comprise a spatial barcode.

In some aspects, provided herein is a composition, comprising: (i) a substrate comprising a uniform lawn of oligonucleotide molecules immobilized on the substrate, wherein the oligonucleotide molecules comprise a common capture region, and (ii) a plurality of circularized probes comprising a common primer binding sequence, wherein the circularized probes are bound to the immobilized oligonucleotide molecules via hybridization of the common primer binding sequence to the common capture region. In some embodiments, the immobilized oligonucleotide molecules are at least 25 nucleotides in length. In any of the preceding embodiments, the immobilized oligonucleotide molecules may be at least 50, 60, 70, or 75 or more nucleotides in length. In any of the preceding embodiments, the capture region may be between about 15 and about 25 nucleotides in length. In any of the preceding embodiments, the immobilized oligonucleotide molecules may comprise a spacer sequence of between about 5 and about 50 nucleotides in length between the capture region and an immobilized end of the oligonucleotide molecules. In any of the preceding embodiments, the immobilized oligonucleotide molecules may but do not need to comprise a spatial barcode.

In some aspects, provided herein is a composition, comprising: (i) a substrate comprising a uniform lawn of oligonucleotide molecules immobilized on the substrate, wherein the immobilized oligonucleotide molecules comprise a common primer sequence, and (ii) a plurality of circularized probes comprising a common primer binding sequence, wherein the circularized probes are bound to the immobilized oligonucleotide molecules via binding of the common primer binding sequence to the common primer sequence. In some embodiments, the immobilized oligonucleotide molecules are at least 25 nucleotides in length. In any of the preceding embodiments, the immobilized oligonucleotide molecules may be at least 50, 60, 70, or 75 or more nucleotides in length. In any of the preceding embodiments, the primer sequence may be between about 15 and about 25 nucleotides in length. In any of the preceding embodiments, at least one of the immobilized oligonucleotide molecules may comprise a spacer sequence of between about 5 and about 50 nucleotides in length between the primer sequence and an immobilized end of the oligonucleotide molecules. In any of the preceding embodiments, the immobilized oligonucleotide molecules may but do not need to comprise a spatial barcode.

In some aspects, provided herein is a kit, comprising: (i) a substrate comprising a uniform lawn of oligonucleotide molecules immobilized on the substrate, wherein the immobilized oligonucleotide molecules comprise a common capture region, and (ii) a plurality of circularizable probes or probe sets, wherein each circularizable probe or probe set comprises a hybridization region complementary to a target nucleic acid sequence, a barcode sequence, and a primer binding sequence, wherein the primer binding sequence is a common primer binding sequence among the plurality of circularizable probes or probe sets; and (iii) instructions for performing the method according to any one of the preceding methods.

In some aspects, provided herein is a kit, comprising: (i) a substrate comprising a uniform lawn of oligonucleotide molecules immobilized on the substrate, wherein the immobilized oligonucleotide molecules comprise a common primer sequence, and (ii) a plurality of circularizable probes or probe sets, wherein each circularizable probe or probe set comprises a binding region complementary to a target nucleic acid sequence, a barcode sequence, and a primer binding sequence, wherein the primer binding sequence is a common primer binding sequence among the plurality of circularizable probes or probe sets; and (iii) instructions for performing the method according to any one of the preceding methods.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The drawings illustrate certain features and advantages of this disclosure. These embodiments are not intended to limit the scope of the appended claims in any manner.

FIGS. 1A-1E depict an exemplary work flow for detecting target nucleic acid using on-substrate rolling circle amplification. As shown in FIG. 1A, a biological sample is placed on a substrate with immobilized oligonucleotide molecules, and circularizable probes (or circularizable probe sets) hybridize to target nucleic acids in the biological sample. The circularizable probes or probe sets are ligated in the biological sample to generate circularized probes, as shown in FIG. 1B. The biological sample is then permeabilized (e.g., digested) as shown in FIG. 1C, and the circularized probes are captured by the oligonucleotide molecules on the substrate as shown in FIG. 1D. Rolling circle amplification of the circularized probes is performed on the substrate to generate rolling circle amplification products as shown in FIG. 1E. The immobilized oligonucleotide molecules comprise primer sequences at their 3′ ends, and by virtue of their hybridization to the circularized probes, function as capture regions to capture the circularized probes from the biological sample.

FIGS. 2A-2E depict an exemplary transfer process (e.g., sandwiching) for detecting target nucleic acid using on-substrate rolling circle amplification. As shown in FIG. 2A, a biological sample is placed on a first substrate, and does not come into direct contact with oligonucleotide molecules immobilized on a second substrate, and circularizable probes or probe sets hybridize to target nucleic acids in the biological sample. The circularizable probes are ligated in the biological sample to generate circularized probes, as shown in FIG. 2B. The biological sample is then permeabilized (e.g., digested) as shown in FIG. 2C, and the circularized probes are migrated to the second substrate and captured by the oligonucleotide molecules on the second substrate as shown in FIG. 2D. Rolling circle amplification of the circularized probes is performed on the second substrate to generate rolling circle amplification products as shown in FIG. 2E.

FIG. 3 depicts the results of an experiment capturing 18S rRNA with immobilized oligonucleotide molecules with reverse complementary sequences on the surface and then extension using RNA dependent DNA polymerase.

FIG. 4 shows the effect of transferring time on capture of circularized probes.

FIG. 5 shows the effect of a crowding agent during rolling circle amplification. Rolling circle amplification products are compact, more uniform in size, and bright.

FIGS. 6A-6B show crowding agents affect the size and signal intensity of rolling circle amplification (RCA) products. FIG. 6A shows that compared to a sample with no crowding agent, adding a crowding agent helps yielding RCA products that are smaller and more uniform in size. FIG. 6B shows adding a crowding agent in 90-minutes RCA generated RCA products on a capture side that are uniform in size and brightness compared to products generated in tissue.

FIG. 7 shows results of detecting RCA products generated on-substrate using captured circularized probes, where the probes were circularized in samples stained with DAPI (post-IF sample) or Hematoxylin & Eosin (post-H&E sample).

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

In situ methods based on rolling circle amplification (RCA) provide a high-throughput solution for detection of target nucleic acids in a biological sample. However, RCA-based methods performed within biological samples are associated with certain drawbacks. For instance, autofluorescence can be severe in some tissue types (such as FFPE tissues). Additionally, tissues are prone to detaching from the substrate, which could cause the signals to come out of focus during decoding of barcodes, which sometimes requires multiple cycles of probe hybridization, detection, and removal to optically resolve a large number of analytes using a limited number of fluorescent channels. In some aspects, the present disclosure provides methods and compositions for performing RCA outside of a biological sample while providing information regarding the location of the target nucleic acids at their positions in the biological sample. In some aspects, provided herein is a targeted approach using probe capture. In some aspects, capturing circularized probes using a uniform lawn of immobilized oligonucleotides with a common capture region (e.g., primer sequence) and then using the capture region as a primer for RCA provides RCA products with high positional stability and mitigates autofluorescence observed in biological samples.

In some aspects, the methods disclosed herein comprise 1) contacting the biological sample with circularizable probes or probe sets that target and hybridize to one or more target nucleic acids in the biological sample; 2) ligating the circularizable probes or probe sets to generate circularized probes at a location in the biological sample; 3) transferring the circularized probes from the biological sample to a substrate comprising a lawn of immobilized oligonucleotide molecules, wherein the oligonucleotide molecules capture the circularized probes; 4) performing RCA on the substrate, thereby generating rolling circle amplification products immobilized on the substrate; and 5) detecting rolling circle amplification products. The methods can further comprise imaging the biological sample prior to transferring the circularized probes, and the imaging data can be overlaid with the detection data, such that the localization of the target nucleic acid in the originating sample is detected. The present application provides working examples demonstrating that a high density lawn of immobilized oligonucleotide molecules comprising primer sequences complementary to a common primer binding sequence of a plurality of circularized probes can efficiently limit lateral diffusion of circularized probes during probe capture, preserving spatial information from the locations of the circularized probes (and corresponding target sequences) in the biological sample.

In some aspects, the methods disclosed herein can reduce autofluorescence and the risk of the biological sample detaching from the substrate by removing the biological sample before the RCA step and the subsequent detection. Location information can still be obtained even though RCA is not performed in situ in the biological sample. The biological sample can be imaged prior to digesting and/or removing the biological sample, and spatial locations of detected RCPs on the substrate can be correlated with positions in the biological sample (e.g., by overlaying data for the RCPs with data obtained from imaging the biological samples). Furthermore, rolling circle amplification products generated on the substrate can be more compact and uniform in size and brightness. In some aspects, rolling circle amplification products generated on the substrate (e.g., after probe capture as described herein) compared to in tissue amplification may exhibit improved characteristics e.g., reduced size. In some cases, removing the tissue may allow substrates and byproducts to have an improved diffusion rate (e.g., more similar to in-vitro reaction).

In some aspects, the methods disclosed herein comprise providing a substrate with a lawn of oligonucleotide molecules immobilized. In some embodiments, the oligonucleotide molecules comprise a primer region that captures at least a portion of the circularized probes. In some embodiments, the biological sample is placed on the substrate. In some embodiments, the biological sample is placed on a first substrate, and a second substrate comprises a lawn of immobilized oligonucleotide molecules. In some aspects, the methods disclosed herein comprise permeabilizing the biological sample. Immobilized oligonucleotide molecules and methods for migrating and capturing circularized probes are described in greater detail in Sections II and III.

In some aspects, the methods disclosed herein comprise staining and imaging the biological sample. In some embodiments, the imaging data is analyzed in combination of detecting of rolling circle amplification products generated by RCA on substrate, thereby providing localization information of the target nucleic acids. Methods for staining and imaging biological samples and detecting rolling circle amplification products on the substrate are described in greater detail in Section III.

### II. Substrate and Immobilized Oligonucleotides

In some aspects, provided herein is a substrate comprising a plurality of oligonucleotide molecules immobilized on the substrate, wherein the immobilized oligonucleotides comprise a capture region (e.g., primer sequence) complementary to a common primer binding sequence in a circularized probe. By hybridization of the capture region (e.g., primer sequence) to the primer binding region, immobilized oligonucleotide molecules can “capture” the circularized probe that is migrated out of the biological sample to the substrate. The capture region (e.g., primer sequence) is then used as a primer for rolling circle amplification of the captured circularized probe (e.g., sequences at the 3′ ends of oligonucleotide molecules immobilized on the substrate in the capture region or a portion thereof function as a primer sequence).

In some embodiments, the substrate comprises a uniform lawn of oligonucleotide molecules immobilized thereon. The immobilized oligonucleotide molecules comprise a capture region (e.g., primer sequence) designed to hybridize to a primer-binding sequence of a circularized probe and act as a primer for rolling circle amplification. In some embodiments, the primer-binding sequence is common among a plurality of circularized probes (e.g., circularized probes that hybridize to different target nucleic acid sequences in the biological sample). In some embodiments, 100% of the capture area is uniformly coated with immobilized oligonucleotide molecules comprising the capture region (e.g., primer sequence). In some cases, the immobilized oligonucleotides are not patterned on the substrate or capture area.

Oligonucleotides may be attached to the substrate according to the methods set forth in U.S. Pat. Nos. 6,737,236, 7,259,258, 7,375,234, 7,427,678, 5,610,287, 5,807,522, 5,837,860, and 5,472,881; U.S. Patent Application Publication Nos. 2008/0280773 and 2011/0059865; Shalon et al. (1996) Genome Research, 639-645; Rogers et al. (1999) Analytical Biochemistry 266, 23-30; Stimpson et al. (1995) Proc. Natl. Acad. Sci. USA 92, 6379-6383; Beattie et al. (1995) Clin. Chem. 45, 700-706; Lamture et al. (1994) Nucleic Acids Research 22, 2121-2125; Beier et al. (1999) Nucleic Acids Research 27, 1970-1977; Joos et al. (1997) Analytical Biochemistry 247, 96-101; Nikiforov et al. (1995) Analytical Biochemistry 227, 201-209; Timofeev et al. (1996) Nucleic Acids Research 24, 3142-3148; Chrisey et al. (1996) Nucleic Acids Research 24, 3031-3039; Guo et al. (1994) Nucleic Acids Research 22, 5456-5465; Running and Urdea (1990) BioTechniques 8, 276-279; Fahy et al. (1993) Nucleic Acids Research 21, 1819-1826; Zhang et al. (1991) 19, 3929-3933; and Rogers et al. (1997) Gene Therapy 4, 1387-1392, all of which are herein incorporated by reference in their entireties.

The oligonucleotide molecules can be attached to a substrate or feature using a variety of techniques. In some embodiments, the oligonucleotide molecules are immobilized to a substrate by chemical immobilization. For example, a chemical immobilization can take place between functional groups on the substrate and corresponding functional elements on the oligonucleotide molecules. Exemplary corresponding functional elements in the oligonucleotide molecules can either be an inherent chemical group of the oligonucleotide molecule, e.g. a hydroxyl group, or a functional element can be introduced on to the oligonucleotide molecule. An example of a functional group on the substrate is an amine group or an N-Hydroxysuccinimide (NHS) ester. In some embodiments, the oligonucleotide molecule to be immobilized includes a functional amine group or is chemically modified in order to include a functional amine group. In some embodiments, the substrate comprising the oligonucleotide molecules is manufactured by dipping the substrate (e.g., the substrate comprising a functional group coating) in an oligonucleotide solution (e.g., a functionalized oligonucleotide solution). In some embodiments, the oligonucleotide molecules are uniformly distributed on the substrate. In some embodiments, the oligonucleotide molecules are uniformly distributed on a capture area of the substrate. In some embodiments, the oligonucleotides completely cover the capture area on the substrate. In some embodiments, functionalized oligonucleotide molecules are immobilized on a functionalized substrate using covalent methods. Methods for covalent attachment include, for example, condensation of amines and activated carboxylic esters (e.g., N-hydroxysuccinimide esters); condensation of amine and aldehydes under reductive amination conditions; and cycloaddition reactions such as the Diels-Alder [4+2] reaction, 1,3-dipolar cycloaddition reactions, and [2+2] cycloaddition reactions. Methods for covalent attachment also include, for example, click chemistry reactions, including [3+2] cycloaddition reactions (e.g., Huisgen 1,3-dipolar cycloaddition reaction and copper(I)-catalyzed azide-alkyne cycloaddition (CuAAC)); thiol-ene reactions; the Diels-Alder reaction and inverse electron demand Diels-Alder reaction; [4+1] cycloaddition of isonitriles and tetrazines; and nucleophilic ring opening of small carbocycles (e.g., epoxide opening with amino oligonucleotides). Methods for covalent attachment also include, for example, maleimides and thiols; and para-nitrophenyl ester-functionalized oligonucleotides and polylysine-functionalized substrate. Methods for covalent attachment also include, for example, disulfide reactions; radical reactions (see, e.g., U.S. Pat. No. 5,919,626, the content of which is herein incorporated by reference in its entirety); and hydrazide-functionalized substrate (e.g., wherein the hydrazide functional group is directly or indirectly attached to the substrate) and aldehyde-functionalized oligonucleotides (see, e.g., Yershov et al. (1996) Proc. Natl. Acad. Sci. USA 93, 4913-4918, the content of which is herein incorporated by reference in its entirety). In some embodiments, the substrate comprising the oligonucleotide molecules is manufactured by dipping the substrate (e.g., the substrate comprising a functional group coating) in an oligonucleotide solution (e.g., a functionalized oligonucleotide solution).

In some embodiments, functionalized oligonucleotide molecules are immobilized on a functionalized substrate using photochemical covalent methods. Methods for photochemical covalent attachment include, for example, immobilization of antraquinone-conjugated oligonucleotides (see, e.g., Koch et al. (2000) Bioconjugate Chem. 11, 474-483, the content of which is herein incorporated by reference in its entirety). In some embodiments, the substrate comprising the oligonucleotide molecules is manufactured by dipping the substrate (e.g., the substrate comprising a functional group coating) in an oligonucleotide solution (e.g., a functionalized oligonucleotide solution).

In some embodiments, functionalized oligonucleotide molecules are immobilized on a functionalized substrate using non-covalent methods. Methods for non-covalent attachment include, for example, biotin-functionalized oligonucleotides and streptavidin-treated substrates (see, e.g., Holmstrom et al. (1993) Analytical Biochemistry 209, 278-283 and Gilles et al. (1999) Nature Biotechnology 17, 365-370, all of which are herein incorporated by reference in their entireties). In some embodiments, the substrate comprising the oligonucleotide molecules is manufactured by dipping the substrate (e.g., the substrate comprising a functional group coating) in an oligonucleotide solution (e.g., a functionalized oligonucleotide solution).

In any of the embodiments herein, the 3′ terminal nucleotides of the immobilized oligonucleotide molecules can be distal to the substrate. In any of the embodiments herein, the 5′ terminal nucleotides of the immobilized oligonucleotide molecules can be more proximal to the substrate than the 3′ terminal nucleotides. In any of the embodiments herein, one or more nucleotides at or near the 5′ terminus of each immobilized oligonucleotide can be directly or indirectly attached to the substrate, thereby immobilizing the oligonucleotides. In some embodiments, the 3′ terminus of each immobilized oligonucleotide is the end that is not immobilized on the substrate. In some embodiments, the oligonucleotide molecules are immobilized on the substrate via a 5′ amino group. In some embodiments, the oligonucleotide molecules comprise a free 3′ hydroxyl group.

In some embodiments, the oligonucleotide molecules comprise a primer region. In some embodiments, the primer region comprises a primer sequence that is complementary to a primer binding sequence in a circularized probe disclosed herein. In some embodiments, the primer region is common to the oligonucleotide molecules. In some embodiments, the primer region is used to capture circularized probes. In some embodiments, the primer region is at the 3′ terminus of the oligonucleotide molecules.

In some embodiments, on a substrate comprising a plurality of oligonucleotide molecules immobilized thereon, at least or about 50%, at least or about 55%, at least or about 60%, at least or about 65%, at least or about 70%, at least or about 75%, at least or about 80%, at least or about 85%, at least or about 90%, at least or about 95%, at least or about 99%, or about 100% of the immobilized oligonucleotide molecules each comprises a primer sequence for rolling circle amplification using a captured circularized probe as a template. In some embodiments, the immobilized oligonucleotide molecules are distributed over a capture area on the substrate, where the capture area corresponds to an area aligned with the biological sample. In some embodiments, at least or about 50%, at least or about 55%, at least or about 60%, at least or about 65%, at least or about 70%, at least or about 75%, at least or about 80%, at least or about 85%, at least or about 90%, at least or about 95%, at least or about 99%, or about 100% of the immobilized oligonucleotide molecules in a capture area each comprises a primer sequence for rolling circular amplification using a captured circularized probe as a template.

In some embodiments, the primer region comprises at least 10 nucleotides. In some embodiments, the primer region comprises 10-40 nucleotides, In some embodiments, the primer region comprises 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 30 nucleotides, 35 nucleotides, or 40 nucleotides.

In some aspects, the length of the primer region relates to the hybridization condition and the preferred melting temperature (Tm) of the sequence. Several equations for calculating the Tm of nucleic acids may be suitable. As indicated by standard references, a simple estimate of the Tm value may be calculated by the equation, Tm=81.5+0.41 (% G+C), when a nucleic acid is in aqueous solution at 1 M NaCl (see e.g., Anderson and Young, Quantitative Filter Hybridization, in Nucleic Acid Hybridization (1985), the content of which is herein incorporated by reference in its entirety). Other references (e.g., Allawi and SantaLucia, Jr., Biochemistry, 36:10581-94 (1997), the content of which is herein incorporated by reference in its entirety) include alternative methods of computation which take structural and environmental, as well as sequence characteristics into account for the calculation of Tm. In some embodiments, the Tm is between any of about 30° C. and about 90° C., about 30° C. and about 80° C., about 50° C. and about 80° C., about 50° C. and about 70° C., about 30° C. and about 32° C., about 32° C. and about 34° C., about 34° C. and about 36° C., about 36° C. and about 38° C., or about 38° C. and about 40° C. In some embodiments, the Tm is optimized based on various conditions, such as the concentrations of DMSO, formamide, salts, and/or primers.

In some embodiments, the immobilized oligonucleotide molecules further comprise linkers (e.g., linkers between the capture region (e.g., primer sequence) and the substrate). In some embodiments, the linker is a nucleic acid sequence of any one of about 10, about 20, about 30, about 40, about 50, or about 60 nucleotides in length, or any range between any of said values. In some cases, the linker is a nucleic acid sequence of between about 5 and about 20, between about 5 and about 30, between about 5 and about 40, between about 10 and about 25, between about 10 and about 35, between about 10 and about 50, between about 10 and about 60, or between about 10 and about 100 nucleotides in length.

In some embodiments, the linker is an organic linker. Exemplary organic linkers include but are not limited to poly-glycols. In some instances, the linker is an alkyl linker or an oligoethylene glycol linker. In some cases, the linker is a tetraethylene glycol, hexaethylene glycol, or decaethylene glycol linker. In some embodiments, the linker is a hydrophilic linker.

In some cases, the linker is a cleavable linker. In some embodiments, the linker is a photocleavable linker, a UV-cleavable linker, an enzyme-cleavable linker, or a pH-sensitive cleavable linker.

In some embodiments, the immobilized oligonucleotides comprise one or more endonuclease-resistant linkages (e.g., any non-natural nuclease resistant internucleoside linkages, such as phosphoramadite linkages). In some embodiments, the immobilized oligonucleotides comprise one, two, three, or more phosphoramadite linkages. In some cases, the one, two, three, or more phosphoramadite linkages are at the 3′ end. In some embodiments, the endonuclease-resistant linkages improve stability of the oligonucleotide molecules immobilized on the substrate.

In some embodiments, the oligonucleotide molecules immobilized on the substrate do not comprise a spatial barcode. In some instances, spatial barcodes might be used to indicate the location of a oligonucleotide molecule on the substrate. In any of the embodiments disclosed herein, the oligonucleotide molecules immobilized on the substrate may but do not need to comprise a spatial barcode.

In some embodiments, a substrate herein can be any support that is insoluble in aqueous liquid and which allows for positioning of biological samples, immobilized oligonucleotide molecules, reagents (e.g., probes such as capture probes) and/or amplification products (e.g., rolling circle amplification products of captured circularized probes) on the support. In some cases, a first substrate and a second substrate are provided, wherein the second substrate comprises the oligonucleotide molecules immobilized thereon and the biological sample is on the first substrate. Circularized probes can be migrated from the biological sample to the second substrate where they may be captured and amplified using the immobilized oligonucleotide molecules on the second substrate. In other cases, the biological sample is on the same substrate as the same substrate as the immobilized oligonucleotide molecules. In some embodiments, the biological sample is digested prior to rolling circle amplification and detecting the rolling circle amplification products.

In some embodiments, the biological sample is attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose. In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

In some aspects, a substrate functions as a support for direct or indirect attachment of oligonucleotides (e.g., to form a lawn of immobilized oligonucleotide molecules). In addition, in some embodiments, a substrate (e.g., the same substrate or a different substrate) can be used to provide support to a biological sample, particularly, for example, a thin tissue section. Accordingly, a “substrate” is a support that is insoluble in aqueous liquid and which allows for positioning of biological samples, immobilized oligonucleotide molecules, and/or captured circularized probes and rolling circle amplification products generated therefrom on the substrate.

A wide variety of different substrates can be used, as long as the substrate is compatible with the sample and sample processing, the reagents and reactions, and signal detection (e.g., optical imaging such as fluorescence microscopy). A substrate can be any suitable support material. For instance, the first substrate and/or the second substrate can be any solid or semi-solid support upon which a biological sample can be mounted. The first substrate and/or the second substrate can include, but are not limited to, glass, modified and/or functionalized glass, hydrogels, films, membranes, plastics, paper, nylon, ceramics, resins, Zeonor, silica or silica-based materials including silicon and modified silicon, carbon, metals, inorganic glasses, optical fiber bundles, and polymers, such as polystyrene, cyclic olefin copolymers (COCs), cyclic olefin polymers (COPs), polypropylene, polyethylene, polycarbonate, and polymer monoliths. In some embodiments, the first substrate and/or the second substrate comprises an inert material or matrix (e.g., glass slides) that has been functionalized by, for example, treating the substrate with a material comprising reactive groups which facilitate mounting of the biological sample. The first substrate and/or the second substrate comprises a substantially flat planar surface. The first substrate and/or the second substrate can be a slide, e.g., a glass slide. For example, a glass slide such as a cover slip may be used. The first substrate and/or the second substrate can be transparent. The first substrate and/or the second substrate can also correspond to or be part of a flow cell. Flow cells can be formed of any of the foregoing materials, and can include channels that permit reagents, solvents, and molecules to pass through the flow cell.

In some instances, a substrate herein (e.g., a first or second substrate) is between about 0.01 mm and about 5 mm, e.g., between about 0.05 mm and about 3 mm, between about 0.1 mm and about 2.5 mm, between about 0.2 mm and about 2 mm, between about 0.5 mm and about 1.5 mm, or about 1 mm in thickness. In some embodiments, the substrate (e.g., a first substrate or a second substrate) is or is about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 mm in thickness, or of a thickness in between any of the aforementioned values.

Among the examples of substrate materials discussed above, polystyrene is a hydrophobic material suitable for binding negatively charged macromolecules because it normally contains few hydrophilic groups. For nucleic acids immobilized on glass slides, by increasing the hydrophobicity of the glass surface the nucleic acid immobilization can be increased. Such an enhancement can permit a relatively more densely packed formation (e.g., provide improved specificity and resolution).

In some embodiments, a substrate is coated with a surface treatment such as poly(L)-lysine. Additionally or alternatively, the substrate can be treated by silanation, e.g. with epoxy-silane, amino-silane, and/or by a treatment with polyacrylamide.

The substrate can generally have any suitable form or format. For example, the substrate can be flat, curved, e.g. convexly or concavely curved towards the area where the interaction between a biological sample, e.g. tissue sample, and the substrate takes place. In some embodiments, the substrate is a flat, e.g., planar, chip or slide. The substrate can contain one or more patterned surfaces within the substrate (e.g., channels, wells, projections, ridges, divots, etc.).

A substrate can be of any desired shape. For example, a substrate can be typically a thin, flat shape (e.g., a square or a rectangle). In some embodiments, a substrate structure has rounded corners (e.g., for increased safety or robustness). In some embodiments, a substrate structure has one or more cut-off corners (e.g., for use with a slide clamp or cross-table). In some embodiments, where a substrate structure is flat, the substrate structure can be any appropriate type of support having a flat surface (e.g., a chip or a slide such as a microscope slide).

In some embodiments, a substrate includes one or more markings on a surface of the substrate, e.g., to provide guidance for correlating spatial information (e.g., cell boundaries or histology from staining and/or immunohistochemistry) with detected rolling circle amplification products on the substrate. For example, a substrate can be marked with a grid of lines (e.g., to allow the size of objects seen under magnification to be easily estimated and/or to provide reference areas for counting objects). In some embodiments, fiducial markers can be included on the substrate. Such markings can be made using techniques including, but not limited to, printing, sand-blasting, and depositing on the surface.

In some instances, the first substrate having a sample attached thereto does not comprise immobilized oligonucleotide molecules on the first substrate. Instead, the immobilized oligonucleotide molecules comprising capture regions (e.g., primer sequences) are provided on one or more second substrates, to which the biological sample is introduced (optionally after imaging the biological sample). For example, a first substrate comprising a biological sample having previously undergone an in situ assay module involving imaging the biological sample, and a second substrate, e.g., comprising a lawn of immobilized oligonucleotide molecules, may be subjected to a sandwiching process described herein to facilitate molecular interaction and/or transfer of circularized probes from the sample to the second substrate.

In some embodiments, the first substrate and/or the second substrate includes one or more markings on its surface, e.g., to provide guidance for aligning at least a portion of the biological sample with a plurality of capture probes on the second substrate during a sandwich process disclosed herein. For example, the first substrate and/or the second substrate can include a sample area indicator identifying the sample area. In some embodiments, during a sandwiching process described herein, the sample area indicator on the first substrate is aligned with an area of the second substrate comprising a plurality of capture probes. In some embodiments, the first and/or second substrate can include a fiducial mark. In some embodiments, the first and/or second substrate does not comprise a fiducial mark. In some embodiments, the first substrate does not comprise a fiducial mark and the second substrate comprises a fiducial mark. Such markings can be made using techniques including, but not limited to, printing, sand-blasting, and depositing on the surface.

In some embodiments, imaging can be performed using one or more fiducial markers, e.g., objects placed in the field of view of an imaging system which appear in the image produced. Fiducial markers are typically used as a point of reference or measurement scale. Fiducial markers are their uses are described in further detail in, e.g., US 2022/0010367 A1, the content of which is herein incorporated by reference in its entirety.

In some embodiments, the biological sample on the first substrate is stained using any of the methods described herein. In some instances, the biological sample is imaged, capturing the stain pattern created during the stain step. In some instances, the biological sample then is destained prior to the sandwiching process (e.g., migration described in Section III).

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the biological sample may be stained with hematoxylin and eosin (H&E). In some embodiments, the biological sample may be stained with DAPI.

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, the biological sample can be stained using a detectable label (e.g., radioisotopes, fluorophores, chemiluminescent compounds, bioluminescent compounds, and dyes) as described elsewhere herein. In some embodiments, a biological sample is stained using only one type of stain or one technique. In some embodiments, staining includes biological staining techniques such as H&E staining. In some embodiments, staining includes biological staining using hematoxylin. In some embodiments, staining includes identifying analytes using fluorescently-conjugated antibodies, e.g., by immunofluorescence. In some embodiments, a biological sample is stained using two or more different types of stains, or two or more different staining techniques. For example, a biological sample can be prepared by staining and imaging using one technique (e.g., H&E staining and brightfield imaging), followed by staining and imaging using another technique (e.g., IHC/IF staining and fluorescence microscopy) for the same biological sample. In some instances, a biological sample on the first substrate is stained.

In some instances, methods for immunofluorescence include a blocking step. The blocking step can include the use of blocking probes to decrease unspecific binding of the antibodies. The blocking step can optionally further include contacting the biological sample with a detergent. In some instances, the detergent can include Triton X100™. The method can further include an antibody incubation step. In some embodiments, the antibody incubation step effects selective binding of the antibody to antigens of interest in the biological sample. In some embodiments, the antibody is conjugated to an oligonucleotide (e.g., an oligonucleotide-antibody conjugate as described herein). In some embodiments, the antibody is not conjugated to an oligonucleotide. In some embodiments, the method further comprises an antibody staining step. The antibody staining step can include a direct method of immunostaining in which a labelled antibody binds directly to the analyte being stained for. Alternatively, the antibody staining step can include an indirect method of immunostaining in which a first antibody binds to the analyte being stained for, and a second, labelled antibody binds to the first antibody. In some embodiments, the antibody staining step is performed prior to sandwich assembly. In some embodiments wherein an oligonucleotide-antibody conjugate is used in the antibody incubation step, the method does not comprise an antibody staining step.

In some instances, the methods include imaging the biological sample. In some instances, imaging occurs prior to sandwich assembly. In some instances, imaging occurs while the sandwich configuration is assembled. In some instances, imaging occurs during permeabilization of the biological sample. In some instances, image are captured using high resolution techniques (e.g., having 300 dots per square inch (dpi) or greater). For example, images can be captured using brightfield imaging (e.g., in the setting of hematoxylin or H&E stain), or using fluorescence microscopy to detect adhered labels. In some instances, high resolution images are captured temporally using e.g., confocal microscopy. In some instances, a low resolution image is captured. A low resolution image (e.g., images that are about 72 dpi and normally have an RGB color setting) can be captured at any point of the workflow, including but not limited to staining, destaining, permeabilization, sandwich assembly, and migration of the analytes. In some instances, a low resolution image is taken during permeabilization of the biological sample.

In some embodiments, the location of the one or more additional analytes in a biological sample are determined by immunofluorescence. In some embodiments, one or more detectable labels (e.g., fluorophore-labeled antibodies, nucleic acid probes disclosed herein) bind to the one or more analytes that are captured (hybridized to) by a probe on the first slide and the location of the one or more analytes is determined by detecting the labels under suitable conditions. In some embodiments, one or more fluorophore-labeled antibodies are used to conjugate to a moiety that associates with a probe on the first slide or the analyte that is hybridized to the probe on the first slide. In some instances, the location(s) of the one or more analytes is determined by imaging the fluorophore-labeled antibodies when the fluorophores are excited by a light of a suitable wavelength. In some embodiments, the location of the one or more analytes in the biological sample is determined by correlating the immunofluorescence data to an image of the biological sample. In some instances, the tissue is imaged throughout the permeabilization step.

In some instances, the biological samples can be destained. In some instances, destaining occurs prior to permeabilization of the biological sample. By way of example only, H&E staining can be destained by washing the sample in HCl. In some instances, the hematoxylin of the H&E stain is destained by washing the sample in HCl. In some embodiments, destaining can include 1, 2, 3, or more washes in HCl. In some embodiments, destaining can include adding HCl to a downstream solution (e.g., permeabilization solution).

### III. Methods

A. Probe Hybridization and Ligation

In some aspects, provided herein is a targeted approach to detecting analytes in a biological sample including capturing a plurality of circularized probes onto a lawn of immobilized oligonucleotides with a common capture region (e.g., primer sequence). In some aspects, the circularized probe is a ligation product of a circularizable probe or probe set. A circularizable probe or probe set can be any nucleic acid molecule or set of nucleic acid molecules that hybridizes to another one or more other nucleic acids such that the ends of the nucleic acid molecule or nucleic acid molecules are juxtaposed or are in proximity for ligation to form a circularized probe (e.g., by ligation with or without gap filling). For example, a circularizable probe may be a padlock probe with ends that can be ligated upon hybridization to a target nucleic acid sequence to form a circularized padlock probe. Specific probe designs can vary depending on the application. For instance, probes or probe sets described herein can comprise a circularizable probe or probe set that does not require gap filling to circularize upon hybridization to a template (e.g., a target nucleic acid and/or a probe such as a splint), a gapped circularizable probe or probe set (e.g., one that requires gap filling to circularize upon hybridization to a DNA template).

In some embodiments, the circularizable probe is provided as a single nucleic acid molecule with ligatable 3′ and 5′ ends. In some cases, the circularizable probe or probe set is provided as a circularizable probe set in two or more parts, e.g. two, three, four, five or more nucleic acid molecules (a probe set), which create two or more ligation junctions upon hybridization to a target nucleic acid and/or splint. In some embodiments, the method comprises contacting the biological sample with a splint. In some embodiments, a first ligation junction is formed between a first and second probe of a circularizable probe set upon hybridization to a target nucleic acid, and a second ligation junction is formed between the first and second probe upon hybridization of the first and second probe to a splint. Each part of the probe may provide (e.g. form or comprise) 3′ and/or 5′ ligatable ends which may be juxtaposed for ligation and be ligated. The 5′ phosphate and 3′ hydroxyl can be ligated together upon hybridization to a target nucleic acid sequence. In some embodiments, the target nucleic acid sequence is in an endogenous nucleic acid analyte (e.g., an endogenous DNA or an endogenous RNA molecule, optionally wherein the endogenous RNA is an mRNA). In some embodiments, the target nucleic acid sequence is in a probe bound directly or indirectly to an endogenous nucleic acid molecule (e.g., a DNA probe hybridized to an endogenous nucleic acid analyte). In some cases, the target nucleic acid sequence is in a product of an endogenous analyte (e.g., an amplification product). For instance, in some embodiments, an RNA analyte is reverse transcribed to generate a DNA molecule, and the circularizable probe or probe set then hybridizes to the DNA molecule. In some cases, the target nucleic acid sequence is in or is associated with a labeling agent that binds to a non-nucleic acid analyte. In some cases, the circularizable probe or probe set is provided as one or more DNA molecules. In some embodiments, the circularizable probe or probe set is or comprises a DNA/RNA chimera comprising one or more ribonucleotides. In some embodiments, circularizable probe or probe set comprises one or more ribonucleotides at and/or near a ligatable 3′ end of the circularizable probe or probe set. In some embodiments, the circularizable probe or probe set comprises a ribonucleotide at its 3′ end. Various exemplary circularizable probes or probe sets are described in US 2020/0224244, the content of which is herein incorporated by reference in its entirety.

In some embodiments, a probe or probe set disclosed herein (e.g., a circularizable probe or probe set) can comprise a 5′ flap which may be recognized by a structure-specific cleavage enzyme, e.g. an enzyme capable of recognizing the junction between single-stranded 5′ overhang and a DNA duplex, and cleaving the single-stranded overhang. It will be understood that the branched three-strand structure which is the substrate for the structure-specific cleavage enzyme may be formed by 5′ end of one probe part and the 3′ end of another probe part when both have hybridized to the target nucleic acid molecule, as well as by the 5′ and 3′ ends of a one-part probe. Enzymes suitable for such cleavage include Flap endonucleases (FENS), which are a class of enzymes having endonucleolytic activity and being capable of catalyzing the hydrolytic cleavage of the phosphodiester bond at the junction of single- and double-stranded DNA. Thus, in some embodiments, cleavage of the additional sequence 5′ to the first target-specific binding site is performed by a structure-specific cleavage enzyme, e.g. a Flap endonuclease. Suitable Flap endonucleases are described in Ma et al. 2000. JBC 275, 24693-24700 and in US 2020/022424, the content of which is herein incorporated by reference in its entirety, and may include P. furiosus (Pfu), A. fulgidus (Afu), M. jannaschii (Mja) or M. thermoautotrophicum (Mth). In other embodiments an enzyme capable of recognizing and degrading a single-stranded oligonucleotide having a free 5′ end may be used to cleave an additional sequence (5′ flap) from a structure as described above. Thus, an enzyme having 5′ nuclease activity may be used to cleave a 5′ additional sequence. Such 5′ nuclease activity may be 5′ exonuclease and/or 5′ endonuclease activity. A 5′ nuclease enzyme is capable of recognizing a free 5′ end of a single-stranded oligonucleotide and degrading said single-stranded oligonucleotide. A 5′ exonuclease degrades a single-stranded oligonucleotide having a free 5′ end by degrading the oligonucleotide into constituent mononucleotides from its 5′ end. A 5′ endonuclease activity may cleave the 5′ flap sequence internally at one or more nucleotides. Further, a 5′ nuclease activity may take place by the enzyme traversing the single-stranded oligonucleotide to a region of duplex once it has recognized the free 5′ end, and cleaving the single-stranded region into larger constituent nucleotides (e.g. dinucleotides or trinucleotides), or cleaving the entire 5′ single-stranded region, e.g. as described in Lyamichev et al. 1999. PNAS 96, 6143-6148 for Taq DNA polymerase and the 5′ nuclease thereof. Preferred enzymes having 5′ nuclease activity include Exonuclease VIII, or a native or recombinant DNA polymerase enzyme from Thermus aquaticus (Taq), Thermus thermophilus or Thermus flavus, or the nuclease domain therefrom.

In some embodiments, the circularizable probes or probe sets contain one or more barcodes. In some embodiments, one or more barcodes are indicative of (e.g., correspond to) the identity of an analyte or labeling agent in the biological sample. In some embodiments, one or more barcodes are indicative of a sequence in the analyte nucleic acid, such as a single nucleotide (e.g., SNPs or point mutations), a dinucleotide sequence, a short sequence of about 5 nucleotides in length, or a sequence of any suitable length.

In some embodiments, the circularizable probe or probe set includes one or more barcode sequences. The barcode sequences, if present, may be of any length. If more than one barcode sequence is used, the barcode sequences may independently have the same or different lengths, such as at least 5, at least 10, at least 15, at least 20, at least 25, at least 30, at least 35, at least 40, at least 50 nucleotides in length. In some embodiments, the barcode sequence may be no more than 120, no more than 112, no more than 104, no more than 96, no more than 88, no more than 80, no more than 72, no more than 64, no more than 56, no more than 48, no more than 40, no more than 32, no more than 24, no more than 16, or no more than 8 nucleotides in length. Combinations of any of these are also possible, e.g., the barcode sequence may be between 5 and 10 nucleotides, between 8 and 15 nucleotides, etc.

The barcode sequence may be arbitrary or random. In certain cases, the barcode sequences are chosen so as to reduce or minimize homology with other components in a sample, e.g., such that the barcode sequences do not themselves bind to or hybridize with other nucleic acids suspected of being within the cell or other sample. In some embodiments, between a particular barcode sequence and another sequence (e.g., a cellular nucleic acid sequence in a sample or other barcode sequences in probes added to the sample), the homology may be less than 10%, less than 8%, less than 7%, less than 6%, less than 5%, less than 4%, less than 3%, less than 2%, or less than 1%. In some embodiments, the homology may be less than 20, 19, 18, 17, 16, 15, 14, 13, 12, 11, 10, 9, 8, 7, 6, 5, 4, 3, or 2 bases, and in some embodiments, the bases are consecutive bases.

In some aspects, the methods provided herein comprise contacting a biological sample with circularizable probes or probe sets that hybridize to target nucleic acid sequence in the biological sample, ligating the circularizable probes or probe sets to generate circularized probes at locations in the biological sample, and then transferring the circularized probes from the biological sample to a substrate comprising a plurality of oligonucleotide molecules immobilized on the substrate, and performing rolling circle amplification of the circularized probes using the immobilized oligonucleotides as primers, thereby generating a rolling circle amplification product immobilized at locations on the substrate, and detecting the rolling circle amplification products at the locations on the substrate.

In some embodiments, the method can further comprise prior to the circularizing step, a step of removing molecules of the circularizable probe or probe set that are not bound to the target nucleic acid from the biological sample. In some embodiments, the method comprises removing splint molecules not hybridized to the circularizable probe or probe set. In any of the embodiments herein, the method can comprise ligating the ends of a circularizable probe or probe set hybridized to the target RNA to form a circularized probe. In any of the embodiments herein, the method can comprise ligating the ends of a circularizable probe or probe set hybridized to a splint to form a circularized probe. In some embodiments, a first ligation is performed using the target RNA, and a second ligation is performed using a splint. In some embodiments, a 3′ end and a 5′ end of the circularizable probe or probe set can be ligated using the target nucleic acid (e.g., RNA) as a template.

In some aspects, the circularizable probes or probe sets comprise a hybridization region, wherein the hybridization region on the probe is capable of hybridizing to a hybridization region on the target nucleic acid. In some aspects, the provided methods involve a step of contacting, or hybridizing the circularizable probes or probe sets to a biological sample containing a target nucleic acid in order to form a hybridization complex. Exemplary biological samples are described in Section IV below. In some embodiments, the biological sample is a tissue sample. In some embodiments, the biological sample is a tissue slice or tissue section. In some embodiments, the biological sample is a fresh frozen tissue sample. In some embodiments, the biological sample is a paraffin embedded formalin fixed (FFPE) tissue sample. In some embodiments, the biological sample is permeabilized. In some embodiments, the method comprises decrosslinking and/or pre-permeabilizing the biological sample before contacting the biological sample with the circularizable probes or probe sets. In some embodiments, the biological sample is permeabilized with a low concentration of proteinase K.

In some aspects, the biological sample is on a substrate. In some cases, the tissue sample is on the substrate comprising the immobilized oligonucleotide molecules on a capture area of the substrate. The capture are is an area of the substrate that is aligned with the biological sample, so that circularized probes migrated from the biological sample toward the substrate can be captured by immobilized oligonucleotide molecules in the capture area. In some instances, the tissue sample is on a first substrate and a second substrate is provided comprising the immobilized oligonucleotide molecules on a surface of the second substrate. In some embodiments, the first substrate and/or the second substrate comprises a substantially flat planar surface. The first substrate and/or the second substrate can be a slide, e.g., a glass slide. For example, a glass slide such as a cover slip may be used. In some embodiments, the first substrate and/or the second substrate is/are optically transparent.

The circularizable probes or probe sets hybridize to target nucleic acid sequences in the biological sample. In some embodiments, the method comprises incubating the sample under conditions optimized for hybridization of the circularizable probes or probe sets to their corresponding target nucleic acid sequences in the biological sample. In some embodiments, the circularizable probes or probe sets are hybridized to the target sequences at a temperature between about 30° C. and about 60° C. (e.g., between about 30° C. and about 50° C.). In some embodiments, the method can further comprise prior to the ligating step, a step of removing molecules of the circularizable probe or probe set that are not bound to the target nucleic acid from the biological sample.

In some embodiments, the hybridization comprises the pairing of substantially complementary or complementary nucleic acid sequences between circularizable probes or probe sets and target nucleic acids. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another. In some embodiments, the method comprises hybridizing the circularizable probes or probe sets to target nucleic acid sequences at conditions optimized for hybridization of circularizable probes or probe sets to fully complementary target nucleic acid sequences. In some cases, the method comprises performing one or more post-hybridization washes. In some embodiments, the one or more post-hybridization washes are performed under stringent conditions.

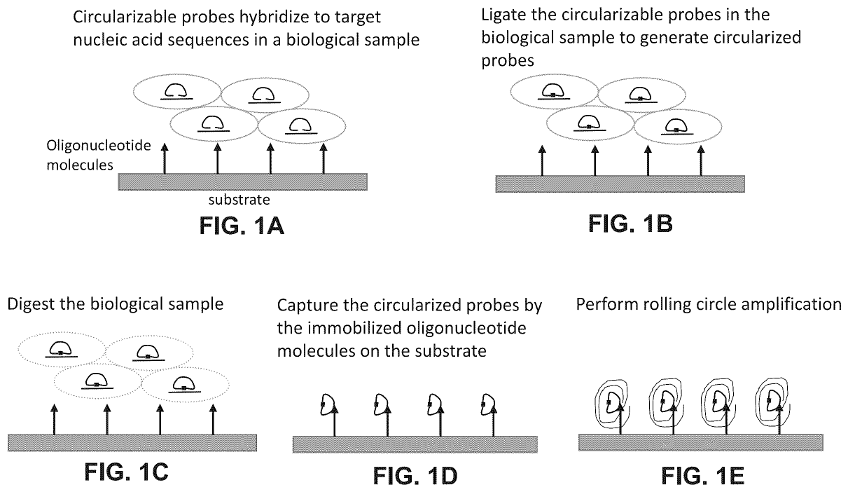

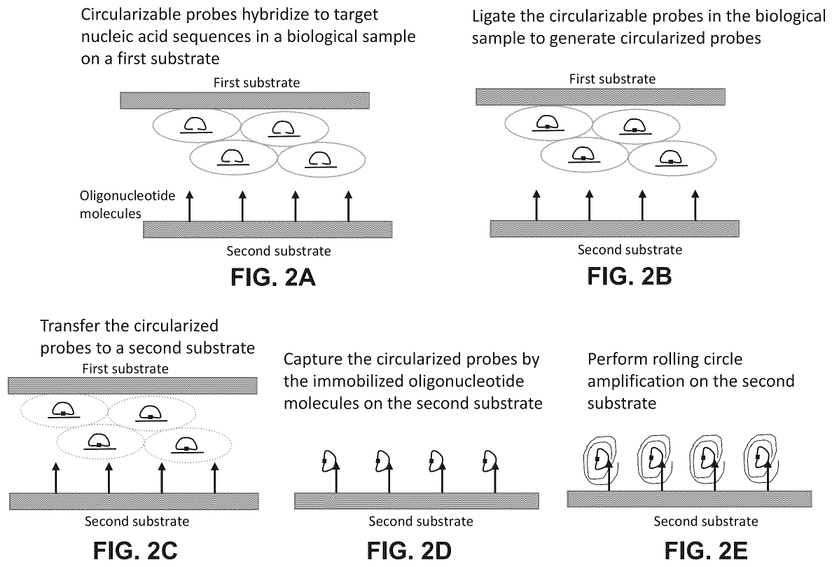

In some aspects, the provided methods comprise one or more steps of ligating a circularizable probe or probe set to form a circularized probe (e.g., as depicted schematically in FIGS. 1B and 2B) at one or more locations in the biological sample. In some embodiments, the ligation involves two or more ligations. In some embodiments, the ligation involves chemical ligation (e.g., click chemistry ligation). In some embodiments, the ligation involves template dependent ligation. In some embodiments, the ligation involves template independent ligation. In some embodiments, the ligation involves enzymatic ligation. In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together.

In some embodiments, the method can further comprise prior to the circularizing step, a step of removing molecules of the circularizable probe or probe set that are not bound to the target nucleic acid from the biological sample. In some embodiments, the method comprises removing splint molecules not hybridized to the circularizable probe or probe set. In any of the embodiments herein, the method can comprise ligating the ends of a circularizable probe or probe set hybridized to the target nucleic acid sequence to form a circularized probe. In any of the embodiments herein, the method can comprise ligating the ends of a circularizable probe or probe set hybridized to a splint to form a circularized probe. In some embodiments, a first ligation is performed using the target nucleic acid sequence, and a second ligation is performed using a splint. In some embodiments, a 3′ end and a 5′ end of the circularizable probe or probe set can be ligated using the target nucleic acid (e.g., RNA) as a template.

In some embodiments, the ligation involves chemical ligation (e.g., click chemistry ligation). In some embodiments, the chemical ligation involves template dependent ligation. In some embodiments, the chemical ligation involves template independent ligation. In some embodiments, the click reaction is a template-independent reaction (see, e.g., Xiong and Seela (2011), J. Org. Chem. 76(14): 5584-5597, incorporated by reference herein in its entirety). In some embodiments, the click reaction is a template-dependent reaction or template-directed reaction. In some embodiments, the template-dependent reaction is sensitive to base pair mismatches such that reaction rate is significantly higher for matched versus unmatched templates. In some embodiments, the click reaction is a nucleophilic addition template-dependent reaction. In some embodiments, the click reaction is a cyclopropane-tetrazine template-dependent reaction.

In some embodiments, the ligation involves enzymatic ligation. In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a direct ligation. In some embodiments, the ligation herein is an indirect ligation. In a direct ligation, the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, e.g., separated by one or more intervening nucleotides or “gaps.” In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap’ corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, circularizable probe, or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, one or more wash steps are performed after ligating the circularizable probes or probe sets to remove unbound probes. In some cases, the one or more wash steps is/are performed under stringent conditions. In some embodiments, the one or more wash steps is/are performed using phosphate buffered saline comprising a non-ionic surfactant. In some embodiments, the wash buffer is PB ST.

In some embodiments, the method comprises digesting or permeabilizing the biological sample. In some embodiments, the method comprises digesting the biological sample after ligating the circularizable probe(s) or probe set(s) using target nucleic acid sequences as templates to form circularized probes. In some cases, the biological sample is digested using a proteinase (e.g., proteinase K). In some embodiments, the biological sample is contacted with an RNase. In some embodiments, circularized probes are released from the target nucleic acid using an endonuclease. In some embodiments, the endonuclease is RNase H. In some cases, the endonuclease digests RNA sequences that are hybridized in DNA/RNA duplexes, thereby releasing the circularized probes from target RNA sequences in the biological sample.

B. Circularized Probe Migration and Capture

In some aspects, the methods provided herein comprise capturing circularized probes using immobilized oligonucleotide molecules on a substrate and using the immobilized oligonucleotide molecules as primers to perform rolling circle amplification of the captured circularized probes, thereby providing a plurality of rolling circle amplification products on the substrate. In some embodiments, the on-substrate rolling circle amplification of circularized probes at positions on the substrate provides a number of advantages, including increased rolling circle amplification efficiency and sensitivity, positional stability of rolling circle amplification products immobilized on the substrate, and/or reduced autofluorescence by detecting amplification products on a substrate rather than in the biological sample. For example, by migrating and capturing the circularized probes, parts of a tissue sample that may contribute to autofluorescence is not contributing to the signals associated with the circularized probes or products thereof. In some examples, less imaging time is needed for the methods provided herein since circularized probes are captured on the surface of the substrate (e.g., reducing or removing the need for imaging in the z-plane). In some cases, the substrate comprises a uniform lawn of immobilized oligonucleotide molecules comprising capture regions (e.g., primer sequences) complementary to a common primer binding region present in a plurality of the circularized probes. The uniform lawn of immobilized oligonucleotide molecules can thus be used to capture circularized probes at positions closely corresponding to the position of the circularized probes in the biological sample prior to migration.

In some aspects, the circularized probes can be captured when contacting the biological sample comprising the circularized probes with a substrate including immobilized oligonucleotide molecules comprising capture regions (e.g., primer sequences) (e.g., a substrate with immobilized oligonucleotide molecules forming a lawn on a capture area of the substrate). As used herein, “contact,” “contacted,” and/or “contacting,” a biological sample with a substrate refers to any contact (e.g., direct or indirect) such that the immobilized oligonucleotide molecules can interact (e.g., bind covalently or non-covalently (e.g., hybridize)) with nucleic acids (e.g., the circularized probes) from the biological sample. Capture can be achieved actively (e.g., using electrophoresis) or passively (e.g., using diffusion).

In some embodiments, a biological sample is provided (e.g., placed) on a substrate comprising a lawn of immobilized oligonucleotide molecules (e.g., as shown in FIG. 1A). In some embodiments, a biological sample is provided (e.g., placed) on a first substrate and a second substrate is provided comprising a lawn of immobilized oligonucleotide molecules (e.g., as shown in FIG. 2A).

In some aspects, after forming circularized probes by template ligation at one or a plurality of ligations in the biological sample, the circularized probes are migrated toward the substrate comprising the immobilized oligonucleotide molecules. In some embodiments, circularized probes can be migrated along an axis substantially perpendicular to the substrate, preserving the original spatial localization of the circularized probes in two-dimensional space. As illustrated schematically in FIG. 1A, in some embodiments, the biological sample is on the substrate comprising a lawn of immobilized oligonucleotide molecules as described above. In some embodiments, the biological sample is permeabilized and/or digested after circularizable probe or probe set ligation (e.g., as illustrated in FIG. 1C). As illustrated in FIG. 1D, in some embodiments circularized probes are captured by the oligonucleotide molecules, wherein the capture region (e.g., primer sequence) comprised by the oligonucleotide molecules binds to the primer binding sequence comprised by the circularized probes. In some embodiments, at least a portion of the circularized probes are captured by hybridization to the capture regions (e.g., primer sequences) of a plurality of the immobilized oligonucleotide molecules.

As illustrated schematically in FIG. 2A, in some embodiments, the biological sample is on a first substrate and the substrate comprising a lawn of immobilized oligonucleotide molecules is a second substrate. The circularized probes can be migrated from the biological sample toward the second substrate, and captured by the oligonucleotide molecules, wherein the capture region (e.g., primer sequence) comprised by the oligonucleotide molecules binds to the primer binding sequence comprised by the circularized probes (FIGS. 2C-2D). In some embodiments, at least a portion of the circularized probes are captured by hybridization to the capture regions (e.g., primer sequences) of a plurality of the immobilized oligonucleotide molecules.

In some embodiments, the biological sample is on a first substrate, and the method comprises migrating the circularized probes toward a second substrate comprising a lawn of immobilized oligonucleotide molecules (e.g., as illustrated in FIGS. 2A-2E). In some embodiments, the circularized probes are transferred to the second substrate without the biological sample, and captured by the oligonucleotide molecules. In some embodiments, the transfer of circularized probes from the first substrate to the oligonucleotide molecules on the second substrate is facilitated by a sandwiching process.

An exemplary embodiment of a workflow for analysis of analytes in a biological sample by a sandwiching process is shown in FIGS. 2A-2E. As shown in FIGS. 2A-2E, a fixed tissue sample mounted on a first substrate (e.g., a slide-mounted tissue sample) is decrosslinked, followed by hybridization of circularizable probes or probe sets to nucleic acid target analytes. Also as shown in FIGS. 2A-2E, a circularizable probe or probe set is circularized, e.g., ligated, at a location in the biological sample. The sample is optionally washed (e.g., with a buffer). In some embodiments, the circularized probes are released from the tissue under sandwich conditions as described herein. For the sandwich conditions, the tissue-mounted slide can be aligned with a second substrate comprising a lawn of immobilized capture oligonucleotide molecules and permeabilized with a reagent medium in the sandwich configuration as described herein. In some embodiments, the reagent medium comprises RNase and a permeabilization agent (e.g., Proteinase K). RNAse releases the circularized probe from a target nucleic acid (such as an RNA analyte), for capture onto a second substrate comprising the lawn of immobilized oligonucleotide molecules. After capture of the circularized probe using the capture region (e.g., primer sequence) of the immobilized oligonucleotide molecules, the first substrate can be removed (e.g., the sandwich can be “opened” or “broken”).

In some embodiments, the first substrate and/or the second substrate includes one or more markings on its surface, e.g., to provide guidance for aligning at least a portion of the biological sample with a plurality of oligonucleotide molecules (e.g., serving as capture probes) on the second substrate during a sandwich process disclosed herein. For example, the first substrate and/or the second substrate can include a sample area indicator identifying the sample area. In some embodiments, during a sandwiching process described herein, the sample area indicator on the first substrate is aligned with an area of the second substrate comprising a plurality of oligonucleotide molecules. In some embodiments, the first and/or second substrate can include a fiducial mark. In some embodiments, the first and/or second substrate does not comprise a fiducial mark. In some embodiments, the first substrate does not comprise a fiducial mark and the second substrate comprises a fiducial mark. Such markings can be made using techniques including, but not limited to, printing, sand-blasting, and depositing on the surface.

In some embodiments, imaging can be performed using one or more fiducial markers, e.g., objects placed in the field of view of an imaging system which appear in the image produced. Fiducial markers are typically used as a point of reference or measurement scale. Fiducial markers are their uses are described in further detail in, e.g., WO 2020/176788 A1, the content of which is incorporated herein by reference in its entirety.

Exemplary substrates similar to the first substrate (e.g., a substrate having no immobilized oligonucleotide molecules for probe capture) and/or the second substrate are described in Section II above. In some embodiments, the sandwiching process may be facilitated by a device, sample holder, sample handling apparatus, or system described in, e.g., US. Patent Application Pub. No. 20210189475, US 2023/0017773 A1, and WO 2022/061152 A2, all of which are herein incorporated by reference in their entireties.

FIGS. 2A-2E depict an exemplary sandwiching process between a first substrate comprising a biological sample (e.g., a tissue section on a slide) and a second substrate comprising a lawn of oligonucleotide molecules. During the exemplary sandwiching process, the first substrate is aligned with the second substrate, such that at least a portion of the biological sample is aligned with at least a portion of the lawn (e.g., aligned in a sandwich configuration). As shown, the first substrate (e.g., slide) may be positioned superior to the second substrate (e.g., slide). In some embodiments, the second substrate (e.g., slide) is in a superior position to the first substrate (e.g., slide). In some embodiments, the biological sample on the first substrate does not come into direct contact with the second substrate (e.g., using one or more spacers). When the first and second substrates are aligned, one or more circularized probes are released from the biological sample and actively or passively migrate to the second substrate for capture. In some embodiments, the migration occurs while the aligned portions of the biological sample and the oligonucleotide lawn are contacted with a reagent that permeabilizes the biological sample, e.g. any permeabilization reagent disclosed herein (FIG. 2C). In some embodiments, the permeabilization reagent is proteinase K and/or RNase H. The released one or more circularized probes may actively or passively migrate towards the second substrate, and be captured by the lawn of oligonucleotide molecules (FIG. 2D).

In some embodiments, the separation distance between first and second substrates is maintained between 2 microns and 1 mm (e.g., between 2 microns and 800 microns, between 2 microns and 700 microns, between 2 microns and 600 microns, between 2 microns and 500 microns, between 2 microns and 400 microns, between 2 microns and 300 microns, between 2 microns and 200 microns, between 2 microns and 100 microns, between 2 microns and 25 microns, between 2 microns and 10 microns), measured in a direction orthogonal to the surface of first substrate that supports sample. In some embodiments, the separation distance between first and second substrates is less than 50 microns. In some instances, the distance is 2 microns. In some instances, the distance is 2.5 microns. In some instances, the distance is about 2, 3, 4, 5, 6, 7, 8, 9, 10, 11, 12, 13, 14, 15, 16, 17, 18, 19, 20, 21, 22, 23, 24, or 25 microns. In some embodiments, the second substrate is placed in direct contact with the sample on the first substrate. In some embodiments, the separation distance is measured in a direction orthogonal to a surface of the first substrate that supports the biological sample.

In some embodiments, the one or more spacers is configured to maintain a separation distance between first and second substrates that is between about 2 microns and 1 mm (e.g., between about 2 microns and 800 microns, between about 2 microns and 700 microns, between about 2 microns and 600 microns, between about 2 microns and 500 microns, between about 2 microns and 400 microns, between about 2 microns and 300 microns, between about 2 microns and 200 microns, between about 2 microns and 100 microns, between about 2 microns and 25 microns, or between about 2 microns and 10 microns), measured in a direction orthogonal to the surface of first substrate that supports the sample. In some instances, the separation distance is about 2, 3, 4, 5, 6, 7, 8, 9, 10, 11, 12, 13, 14, 15, 16, 17, 18, 19, 20, 21, 22, 23, 24, or 25 microns. In some embodiments, the separation distance is less than 50 microns. In some embodiments, the separation distance is less than 25 microns. In some embodiments, the separation distance is less than 20 microns. The separation distance may include a distance of at least 2 μm.

In some embodiments, the first and second substrates are placed in a substrate holder (e.g., a biological sample/substrate capture area alignment device). In some embodiments, the device comprises a sample holder. In some embodiments, the sample holder includes a first member and a second member that receive a first substrate and a second substrate, respectively. The device can include an alignment mechanism that is connected to at least one of the members and aligns the first and second members. Thus, the devices of the disclosure can advantageously align the first substrate and the second substrate and any samples, immobilized oligonucleotide molecules, or permeabilization reagents that may be on the surface of the first and second substrates.

In some embodiments, the sandwiching process comprises: mounting the first substrate on a first member of a support device, the first member configured to retain the first substrate; mounting the second substrate on a second member of the support device, the second member configured to retain the second substrate, applying a reagent medium to the first substrate and/or the second substrate, the reagent medium comprising a permeabilization agent, operating an alignment mechanism of the support device to move the first member and/or the second member such that a portion of the biological sample is aligned (e.g., vertically aligned) with a portion of the oligonucleotide molecules on the second substrate and within a threshold distance of the lawn of oligonucleotide molecules, and such that the portion of the biological sample and the capture probe contact the reagent medium, wherein the permeabilization reagent releases the circularized probes from the biological sample.

In some embodiments of a sample holder, the sample holder can include a first member including a first retaining mechanism configured to retain a first substrate comprising a sample. The first retaining mechanism can be configured to retain the first substrate disposed in a first plane. The sample holder can further include a second member including a second retaining mechanism configured to retain a second substrate disposed in a second plane. The sample holder can further includes an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The alignment mechanism may be configured to move the second member along the axis orthogonal to the second plane and/or move the first member along an axis orthogonal to the first plane.

In some embodiments, the alignment mechanism includes a linear actuator. In some embodiments, the alignment mechanism includes one or more of a moving plate, a bushing, a shoulder screw, a motor bracket, and a linear actuator. The moving plate may be coupled to the first member or the second member. The alignment mechanism may, in some cases, include a first moving plate coupled to the first member and a second moving plate coupled to the second member. In some embodiments, the linear actuator is configured to move the second member along an axis orthogonal to the plane or the first member and/or the second member. For example, the moving plate may be coupled to the second member and adjust the separation distance along a z axis (e.g., orthogonal to the second substrate) by moving the moving plate up in a superior direction toward the first substrate. In some embodiments, the linear actuator is configured to move the first member along an axis orthogonal to the plane of the first member and/or the second member. The movement of the moving plate may be accomplished by the linear actuator configured to move the first member and/or the second member at a velocity. The velocity may be controlled by a controller communicatively coupled to the linear actuator. In some embodiments, the linear actuator is configured to move the first member, the second member, or both the first member and the second member at a velocity of at least 0.1 mm/sec (e.g., at least 0.1 mm/sec to 2 mm/sec). In some aspects, the velocity may be selected to reduce or minimize bubble generation or trapping within the reagent medium. In some embodiments, the linear actuator is configured to move the first member, the second member, or both the first member and the second member with an amount of force of at least 0.1 lbs. (e.g., between 0.1-4.0 pounds of force).

In some aspects, the velocity of the moving plate (e.g., closing the sandwich) may affect bubble generation or trapping within the reagent medium. It may be advantageous to minimize bubble generation or trapping within the reagent medium during the “sandwiching” process, as bubbles can interfere with the migration of circularized probes through the reagent medium to the oligonucleotide lawn. In some embodiments, the closing speed is selected to minimize bubble generation or trapping within the reagent medium. In some embodiments, the closing speed is selected to reduce the time it takes the flow front of the reagent medium from an initial point of contact with the first and second substrate to sweep across the sandwich area (also referred to herein as “closing time”). In some embodiments, the closing speed is selected to reduce the closing time to less than about 1100 ms. In some embodiments, the closing speed is selected to reduce the closing time to less than about 1000 ms. In some embodiments, the closing speed is selected to reduce the closing time to less than about 900 ms. In some embodiments, the closing speed is selected to reduce the closing time to less than about 750 ms. In some embodiments, the closing speed is selected to reduce the closing time to less than about 600 ms. In some embodiments, the closing speed is selected to reduce the closing time to about 550 ms or less. In some embodiments, the closing speed is selected to reduce the closing time to about 370 ms or less. In some embodiments, the closing speed is selected to reduce the closing time to about 200 ms or less. In some embodiments, the closing speed is selected to reduce the closing time to about 150 ms or less.

Further details on support devices, sample holders, sample handling apparatuses, or systems for implementing a sandwiching process for probe migration and capture are described in, e.g., US 2022/0241780 A1 and WO 2022/061152 A2, all of which are herein incorporated by reference in their entireties.

In some aspects, it may be possible to reduce or eliminate bubble formation between the slides (e.g., two substrates) using a variety of filling methods and/or closing methods. In some embodiments, the substrates can be closed at an angle. Further details on angled closure workflows, and devices and systems for implementing an angled closure workflow, are described in US 2023/0017773 A1 and WO 2022/061152 A2, all of which are herein incorporated by reference in their entireties.

Additional configurations for reducing or eliminating bubble formation, and/or for reducing unwanted fluid flow, are described in US 2023/0017773 A1, which is herein incorporated by reference in its entirety.

Workflows described herein may include contacting a drop of the reagent medium (e.g., liquid reagent medium, e.g., a permeabilization solution) disposed on a first substrate or a second substrate with at least a portion of the second substrate or first substrate, respectively. In some embodiments, the contacting comprises bringing the two substrates into proximity such that the sample on the first substrate is aligned with the oligonucleotide molecules on the second substrate.

In some embodiments, the reagent medium comprises a permeabilization agent. Suitable agents for this purpose include, but are not limited to, organic solvents (e.g., acetone, ethanol, and methanol), cross-linking agents (e.g., paraformaldehyde), detergents (e.g., saponin, Triton X-100™, Tween-20™, or sodium dodecyl sulfate (SDS)), and enzymes (e.g., trypsin, proteases (e.g., proteinase K). In some embodiments, the detergent is an anionic detergent (e.g., SDS or N-lauroylsarcosine sodium salt solution). Exemplary permeabilization reagents are described in in US. Patent Application Pub. No. 20210189475, which is incorporated by reference in its entirety.

In some embodiments, the reagent medium comprises a lysis reagent. Lysis solutions can include ionic surfactants such as, for example, sarkosyl and sodium dodecyl sulfate (SDS). More generally, chemical lysis agents can include, without limitation, organic solvents, chelating agents, detergents, surfactants, and chaotropic agents. Exemplary lysis reagents are described in US. Patent Application Pub. No. 20210189475, which is incorporated by reference in its entirety.

In some embodiments, the reagent medium comprises a protease. Exemplary proteases include, e.g., pepsin, trypsin, pepsin, elastase, and proteinase K. Exemplary proteases are described in US. Patent Application Pub. No. 20210189475, which is incorporated by reference in its entirety.

In some embodiments, the reagent medium comprises a detergent. Exemplary detergents include sodium dodecyl sulfate (SDS), sarkosyl, saponin, Triton X-100™, and Tween-20™. Exemplary detergents are described in US. Patent Application Pub. No. 20210189475, which is incorporated by reference in its entirety.

In some embodiments, the reagent medium comprises a nuclease. In some embodiments, the nuclease comprises an RNase. In some embodiments, the RNase is selected from RNase A, RNase C, RNase H, and RNase I. In some embodiments, the reagent medium comprises one or more of sodium dodecyl sulfate (SDS), proteinase K, pepsin, N-lauroylsarcosine, RNase, and a sodium salt thereof.

In some embodiments, the reagent medium comprises an agent for releasing a connected probe disclosed herein and a permeabilization agent. In some embodiments, the agent for releasing the connected probe comprises or is a nuclease, e.g., RNase, and the permeabilization agent is a protease (e.g., proteinase K, trypsin, pepsin, elastase).

In some embodiments, the reagent medium comprises polyethylene glycol (PEG). In some embodiments, the PEG is from about PEG 2K to about PEG 16K. In some embodiments, the PEG is PEG 2K, 3K, 4K, 5K, 6K, 7K, 8K, 9K, 10K, 11K, 12K, 13K, 14K, 15K, or 16K. In some embodiments, the PEG is present at a concentration from about 2% to 25%, from about 4% to about 23%, from about 6% to about 21%, or from about 8% to about 20% (v/v).

In some embodiments, the reagent medium includes a wetting agent.

In some instances, the biological sample is in contact with the reagent medium for about 1 minute. In some instances, the biological sample is in contact with the reagent medium for about 5 minutes. In some instances, the biological sample is in contact with the reagent medium in the gap for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the biological sample is in contact with the reagent medium for about 1-60 minutes, about 10-60 minutes, about 30-60 minutes, or about 20-45 minutes. In some instances, the biological sample is in contact with the reagent medium for about 30 minutes. In some instances, the biological sample is in contact with the reagent medium for at least about any of 30, 40, 45, 50, 55, or 60 minutes. In some instances, the biological sample is in contact with the reagent medium for about 60 minutes.

The sample holder is compatible with a variety of different schemes for contacting the aligned portions of the biological sample and substrate with the reagent medium to promote circularized probe capture. In some embodiments, the reagent medium is deposited directly on the second substrate, and/or directly on the first substrate. In some embodiments, the reagent medium is deposited on the first and/or second substrate, and then the first and second substrates aligned in the sandwich configuration such that the reagent medium contacts the aligned portions of the biological sample and substrate. In some embodiments, the reagent medium is introduced into the gap while the first and second substrates are aligned in the sandwich configuration.

In certain embodiments a dried permeabilization reagent is applied or formed as a layer on the first substrate or the second substrate or both prior to contacting the sample and the second substrate. For example, a reagent can be deposited in solution on the first substrate or the second substrate or both and then dried. Drying methods include, but are not limited to spin coating a thin solution of the reagent and then evaporating a solvent included in the reagent or the reagent itself. Alternatively, in other embodiments, the reagent can be applied in dried form directly onto the first substrate or the second substrate or both. In some embodiments, the coating process can be done in advance of the analytical workflow and the first substrate and the second substrate can be stored pre-coated. Alternatively, the coating process can be done as part of the analytical workflow. In some embodiments, the reagent is a permeabilization reagent. In some embodiments, the reagent is a permeabilization enzyme, a buffer, a detergent, or any combination thereof. In some embodiments, the permeabilization enzyme is pepsin. In some embodiments, the reagent is a dried reagent (e.g., a reagent free from moisture or liquid). In some instances, the substrate that includes the sample (e.g., a histological tissue section) is hydrated. The sample can be hydrated by contacting the sample with a reagent medium, e.g., a buffer that does not include a permeabilization reagent. In some embodiments, the hydration is performed while the first and second substrates are aligned in a sandwich configuration.

In some embodiments, the drop includes permeabilization reagents (e.g., any of the permeabilization reagents described herein). In some embodiments, the rate of permeabilization of the biological sample is modulated by delivering the permeabilization reagents (e.g., a fluid containing permeabilization reagents) at various temperatures.

In some instances, the device is configured to control a temperature of the first and second substrates. In some embodiments, the temperature of the first and second members is lowered to a first temperature that is below room temperature (e.g., 25 degrees Celsius) (e.g., 20 degrees Celsius or lower, 15 degrees Celsius or lower, 10 degrees Celsius or lower, 5 degrees Celsius or lower, 4 degrees Celsius or lower, 3 degrees Celsius or lower, 2 degrees Celsius or lower, 1 degree Celsius or lower, 0 degrees Celsius or lower, −1 degrees Celsius or lower, −5 degrees Celsius or lower). In some embodiments, the device includes a temperature control system (e.g., heating and cooling conducting coils) to control the temperature of the sample holder. Alternatively, in other embodiments, the temperature of the sample holder is controlled externally (e.g., via refrigeration or a hotplate). In a first step, the second member, set to or at the first temperature, contacts the first substrate, and the first member, set to or at the first temperature, contacts the second substrate, thereby lowering the temperature of the first substrate and the second substrate to a second temperature. In some embodiments, the second temperature is equivalent to the first temperature. In some embodiments, the first temperature is lower than room temperature (e.g., 25 degrees Celsius). In some embodiments, the second temperature ranges from about −10 degrees Celsius to about 4 degrees Celsius. In some embodiments, the second temperature is below room temperature (e.g., 25 degrees Celsius) (e.g., 20 degrees Celsius or lower, 15 degrees Celsius or lower, 10 degrees Celsius or lower, 5 degrees Celsius or lower, 4 degrees Celsius or lower, 3 degrees Celsius or lower, 2 degrees Celsius or lower, 1 degree Celsius or lower, 0 degrees Celsius or lower, −1 degrees Celsius or lower, −5 degrees Celsius or lower).

In certain embodiments a dried permeabilization reagent is applied or formed as a layer on the first substrate or the second substrate or both prior to contacting the first and the second substrate. For example, a reagent can be deposited in solution on the first substrate or the second substrate or both and then dried. Drying methods include, but are not limited to spin coating a thin solution of the reagent and then evaporating a solvent included in the reagent or the reagent itself. Alternatively, in other embodiments, the reagent can be applied in dried form directly onto the first substrate or the second substrate or both. In some embodiments, the coating process can be done in advance of the analytical workflow and the first substrate and the second substrate can be stored pre-coated. Alternatively, the coating process can be done as part of the analytical workflow. In some embodiments, the reagent is a permeabilization reagent. In some embodiments, the reagent is a permeabilization enzyme, a buffer, a detergent, or any combination thereof. In some embodiments, the permeabilization enzyme is pepsin. In some embodiments, the reagent is a dried reagent (e.g., a reagent free from moisture or liquid). In some instances, the substrate that includes the sample (e.g., a histological tissue section) is hydrated. The sample can be hydrated by contacting the sample with a reagent medium, e.g., a buffer that does not include a permeabilization reagent. In some embodiments, the hydration is performed while the first and second substrates are aligned in a sandwich configuration.

In some instances, the aligned portions of the biological sample and the second substrate (e.g., which is a substrate comprising a plurality of oligonucleotide molecules immobilized on the substrate as described in Section II) are in contact with the reagent medium for about 1 minute. In some instances, the aligned portions of the biological sample and the second substrate are in contact with the reagent medium for about 5 minutes. In some instances, the aligned portions of the biological sample and the second substrate are in contact with the reagent medium in the gap for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the aligned portions of the biological sample and the second substrate are in contact with the reagent medium for about 1-60 minutes. In some instances, the aligned portions of the biological sample and the second substrate are in contact with the reagent medium for about 30 minutes.

In some embodiments, following initial contact between sample and a permeabilization agent, the permeabilization agent can be removed from contact with sample (e.g., by opening sample holder).

In some embodiments, following initial contact between sample and a permeabilization agent, the permeabilization agent can be removed from contact with sample (e.g., by opening sample holder) before complete permeabilization of sample. For example, in some embodiments, only a portion of sample is permeabilized, and only a portion of the analytes in sample may be captured by the immobilized oligonucleotides on the second substrate. In some instances, the reduced amount of circularized probes captured and available for detection can be offset by the reduction in lateral diffusion that results from incomplete permeabilization of sample. In general, the spatial resolution of the assay is determined by the extent of analyte diffusion in the transverse direction (e.g., orthogonal to the normal direction to the surface of sample). The larger the distance between the sample on the first substrate and the immobilized oligonucleotide lawn on the second substrate, the greater the extent of diffusion in the transverse direction, and the concomitant loss of resolution. Analytes liberated from a portion of the sample closest to the immobilized oligonucleotide lawn have a shorter diffusion path, and therefore do not diffuse as far laterally as analytes from portions of the sample farthest from the immobilized oligonucleotide lawn. As a result, in some instances, incomplete permeabilization of the sample (by reducing the contact interval between the permeabilization agent and the sample) can be used to maintain adequate spatial resolution in the assay.

In some embodiments, the workflow includes provision of the first substrate comprising the biological sample. In some embodiments, the workflow includes mounting the biological sample onto the first substrate. In some embodiments wherein the biological sample is a tissue sample, the workflow include sectioning of the tissue sample (e.g., cryostat sectioning). In some embodiments, the workflow includes a fixation step. In some instances, the fixation step can include fixation with methanol. In some instances, the fixation step includes formalin (e.g., 2% formalin).

Between any of the methods disclosed herein, the methods can include a wash step (e.g., with SSC (e.g., 0.1×SSC)). Wash steps can be performed once or multiple times (e.g., 1×, 2×, 3×, between steps disclosed herein). In some instances, wash steps are performed for about 10 seconds, about 15 seconds, about 20 seconds, about 30 seconds, or about a minute. In some instances, three washes occur for 20 seconds each. In some instances, the wash step occurs before staining the sample, after destaining the sample, before permeabilization the sample, after permeabilization the sample, or any combination thereof.

C. On-Substrate Rolling Circle Amplification

In some embodiments, the methods disclosed herein comprise migrating circularized probes out of biological samples and capturing circularized probes on a substrate. In some embodiments, the methods disclosed herein comprise performing rolling circle amplification on the substrate using the captured circularized probes.

In some embodiments, the methods disclosed herein comprise performing rolling circle amplification (RCA) to generate a rolling circle amplification product (RCP) in the absence of a target nucleic acid and a biological sample (FIGS. 1E and 2E). In some embodiments, the RCA comprises a linear RCA. In some embodiments, the RCA comprises a branched RCA. In some embodiments, the RCA comprises a dendritic RCA. In some embodiments, the RCA comprises any combination of the foregoing. (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 1 1:1095-1099, 2001; Schweitzer et al, Nature Biotech. 20:359-365, 2002; U.S. Pat. Nos. 6,054,274, 6,291,187, 6,323,009, 6,344,329 and 6,368,801). In some embodiments, the primer region comprised by the oligonucleotide molecules is used as a primer for RCA.

In any of the embodiments herein, the RCA can comprise a linear RCA, a branched RCA, a dendritic RCA, or any combination thereof. In any of the preceding embodiments, the amplification product can be generated using a polymerase selected from the group consisting of Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, and a variant or derivative thereof. In some embodiment, the polymerase is Phi29 DNA polymerase.

In some embodiments, upon addition of a DNA polymerase in the presence of appropriate dNTP precursors and other cofactors, the capture region (e.g., primer sequence) of the an immobilized oligonucleotide primes elongation to produce multiple copies of the circular template (e.g., a circularized probe). This amplification step can utilize isothermal amplification or non-isothermal amplification. In some embodiments, after the formation of the hybridization complex by hybridization of the capture region (e.g., primer sequence) to the primer binding region of the circularized probe, the hybridization complex is rolling-circle amplified to generate a rolling circle amplification product (RCP) containing multiple complementary copies of the circularized probe. Because the immobilized oligonucleotide molecules are extended to form the RCPs, the RCPs will be immobilized on the substrate.

In some embodiments, RCA is performed in a buffer comprising a crowding agent. In some embodiments, the crowding agent is selected from the group comprising poly(ethylene glycol) (PEG), glycerol, Ficoll, and dextran sulfate. In some embodiments, the crowding agent is poly(ethylene glycol) (PEG). the PEG is selected from the group consisting of PEG200, PEG8000, and PEG35000. In some embodiments, the buffer comprises between about 5% and about 15% PEG. In some embodiments, the buffer comprises between about 5% and about 10% dextran sulfate. In some embodiments, the buffer comprises about 10% PEG.

In some embodiments, RCA is performed for no more than 60 minutes. In some embodiments, RCA is performed for no more than 90 minutes. In some embodiments, RCA is performed for no more than 120 minutes.

In some embodiments, RCA is performed at a temperature between or between about 20° C. and about 60° C. In some embodiments, RCA is performed at a temperature between or between about 30° C. and about 40° C. In some aspects, RCA is performed at a temperature between at or about 25° C. and at or about 45° C., such as at or about 25° C., 27° C., 29° C., 31° C., 33° C., 35° C., 37° C., 39° C., 41° C., 43° C., or 45° C.

In some aspects, rolling circle amplification of captured circularized probes using the capture regions (e.g., primer sequences) of immobilized oligonucleotides as primers results in the formation of rolling circle amplification products covalently attached to the immobilized oligonucleotide molecules, thereby providing amplification products immobilized on the substrate. Thus, the methods provided herein can preserve the spatial localization of the rolling circle amplification products on the substrate, which can be correlated with the location in the sample.

In any of the preceding embodiments, the amplification product can be immobilized on the substrate for subsequent processing, detection and analysis.

D. Detection and Analysis

In some aspects, the methods disclosed herein further comprise detecting RCA products (RCPs) immobilized on a substrate. In some cases, the methods may also comprise imaging the biological sample (e.g., on a first substrate prior to transfer of the circularized probes to a second substrate different from the first substrate, or on the same substrate prior to removal of the biological sample). In some embodiments, the method comprises overlaying or superimposing data obtained from imaging the biological sample with data obtained from detecting the rolling circle amplification products on the substrate. For example, in some cases tissue segmentation information (e.g., based on H&E staining or immunofluorescence) is overlaid with the detected RCPs to correlate spatial localizations of RCPs on the substrate with the position of the corresponding analytes in the biological sample. In some cases, one or more fiducial markers are used to help correlate the positions in the biological sample with positions of RCPs on the substrate. In some embodiments, a plurality of images of the biological sample is acquired. For examples, one or more images of the biological sample stained with a nuclear stain, a histological stain, and/or an immunologic stain can be acquired and one or more images can be acquired to detect RCPs in the biological sample. In some aspects, the plurality of images can be acquired separately (e.g., prior to and after capture of the circularized probes).

### (i). Imaging of Biological Samples

In some aspects, the methods provided herein comprise imaging a biological sample. In some instances, an image of a biological sample is analyzed in combination with data from on-substrate detection of amplification products. In some embodiments, the biological sample is imaged prior to contacting the sample with circularizable probes or probe sets. In some embodiments, the biological sample is imaged after contacting the sample with circularizable probes or probe sets. In some embodiments, the biological sample is stained prior to imaging. In some embodiments, the biological sample is stained with a nuclear stain, a histological stain, and/or an immunologic stain. In some embodiments, the biological sample is stained with hematoxylin and eosin (H&E) stain. In some embodiments, the biological sample is stained with DAPI.

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, ruthenium red, propidium iodide, rhodamine (e.g., rhodamine B), or safranine or derivatives thereof. In some embodiments, the sample may be stained with hematoxylin and eosin (H&E). In some embodiments, the biological sample is stained with DAPI.

DAPI (4′,6-diamidino-2-phenylindole) is a DNA-specific probe which forms a fluorescent complex by attaching in the minor grove of A-T rich sequences of DNA. It also forms nonfluorescent intercalative complexes with double-stranded nucleic acids. As DAPI can pass through an intact cell membrane, it can be used to stain both live and fixed cells. When bound to double-stranded DNA, DAPI has an absorption maximum at a wavelength of 358 nm (ultraviolet) and its emission maximum is at 461 nm (blue). Therefore, for fluorescence microscopy, DAPI is excited with ultraviolet light and is detected through a blue/cyan filter.

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Any suitable methods of destaining or discoloring a biological sample may be utilized, and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

In some embodiments, imaging can be performed using one or more fiducial markers, e.g., objects placed in the field of view of an imaging system which appear in the image produced. Fiducial markers are typically used as a point of reference or measurement scale. Fiducial markers are their uses are described in further detail in, e.g., WO 2020/176788 A1, the entire contents of which are incorporated herein by reference. In some embodiments, the one or more fiducial markers are florescent markers.

In some embodiments, the imaging is performed using a microscopy method such as bright field microscopy (e.g., to detect H&E staining), oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, the biological sample is imaged by confocal microscopy (for example, to detect immunofluorescent staining such as DAPI for identifying cell structures including nuclei, protein expression, or cell boundaries). Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data. In some embodiments, the biological sample is imaged by fluorescent microscopy and/or brightfield microscopy.

### (ii). Detecting Rolling Circle Amplification Products

In any of the embodiments herein, the detecting can comprise detecting RCPs in the absence of a biological sample (e.g., generated using circularized probes captured on the substrate). In any of the embodiments herein, the detecting can comprise detecting a signal associated with a fluorescently labeled probe that directly or indirectly binds to a RCP of the circularized probe. In any of the embodiments herein, the sequence of RCP or other generated product can be analyzed by sequential hybridization, sequencing by hybridization, sequencing by ligation, sequencing by synthesis, sequencing by binding, or a combination thereof.

In some embodiments, the RCPs are detected as fluorescent spots on the substrate. In some embodiments, the diameters of at least 80%, at least 90%, or at least 95% of the rolling circle amplification products (RCP) (e.g., the diameters of the detected fluorescent spots) are within 3 μm of the median detected RCP diameter.

In any of the embodiments herein, the detecting step can comprise contacting the substrate with one or more detectably-labeled probes that directly or indirectly bind to the rolling circle amplification product. In some embodiments, the method comprises removing (e.g., dehybridizing) the one or more detectably-labeled probes from the rolling circle amplification product. In any of the embodiments herein, the contacting and removing (e.g., dehybridizing) steps can be repeated with the one or more detectably-labeled probes and/or one or more other detectably-labeled probes that directly or indirectly bind to the rolling circle amplification product.

In some embodiments, the detectably labeled probes bind to one or more intermediate probes comprising one or more overhang regions (e.g., a 5′ and/or 3′ end of the probe that does not hybridize to the rolling circle amplification product). A probe comprising a single overhang region may be referred to as an “L-shaped probe,” and a probe comprising two overhangs may be referred to as a “U-shaped probe.” In some cases, the overhang region comprises a binding region for binding one or more detectably-labeled probes. In some embodiments, the detecting comprises contacting the substrate with a pool of intermediate probes corresponding to different barcode sequences or portions thereof, and a pool of detectably-labeled probes corresponding to different detectable labels. In some embodiments, the substrate is sequentially contacted with different pools of intermediate probes. In some instances, a common or universal pool of detectably-labeled probes is used in a plurality of sequential hybridization steps (e.g., with different pools of intermediate probes).

In some embodiments, in a barcode sequencing method, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4′ complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and U.S. Pat. Pub 20210164039, all of which are herein incorporated by reference in their entireties.

In any of the embodiments herein, the detecting step can comprise contacting the substrate with one or more intermediate probes that directly or indirectly bind to the rolling circle amplification product, wherein the one or more intermediate probes are detectable using one or more detectably-labeled probes. In some embodiments, the intermediate probe comprises: (i) a recognition sequence complementary to a barcode sequence or portion thereof in the rolling circle amplification product, and (ii) a binding site for the detectably labeled probe. In any of the embodiments herein, the detecting step can further comprise removing (e.g., dehybridizing) the one or more intermediate probes and/or the one or more detectably-labeled probes from the rolling circle amplification product. In any of the embodiments herein, the contacting and removing (e.g., dehybridizing) steps can be repeated with the one or more intermediate probes, the one or more detectably-labeled probes, one or more other intermediate probes, and/or one or more other detectably-labeled probes.

In any of the embodiments herein, the detecting of RCPs can be correlated with imaging of the biological sample as described above, to determine the locations of corresponding analytes in the biological sample. In some embodiments, the data can be overlaid with the help of fiducial marks on the substrate.

In some embodiments, fluorescence microscopy is used for detection and imaging of the detectably labeled probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, all or a portion of the RCP (e.g., a barcode sequence in the RCP) is detected by sequencing on the substrate. In some embodiments, sequencing of RCPs on the substrate can be performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218; and 6,306,597, all of which are herein incorporated by reference in their entireties.

In some embodiments, all or a portion of the RCP (e.g., a barcode sequence in the RCP) is detected by sequencing on the substrate, using a base-by-base sequencing method, e.g., SBS or SBB. In some embodiments, the biological sample is contacted with a sequencing primer and base-by-base sequencing using a cyclic series of nucleotide incorporation or binding, respectively, thereby generating extension products of the sequencing primer is performed followed by removing, cleaving, or blocking the extension products of the sequencing primer.

Generally in sequencing-by-synthesis methods, a first population of detectably labeled nucleotides (e.g., dNTPs) are introduced to contact a template nucleotide (e.g., a barcode sequence in the RCP) hybridized to a sequencing primer, and a first detectably labeled nucleotide (e.g., A, T, C, or G nucleotide) is incorporated by a polymerase to extend the sequencing primer in the 5′ to 3′ direction using a complementary nucleotide (a first nucleotide residue) in the template nucleotide as template. A signal from the first detectably labeled nucleotide can then be detected. The first population of nucleotides may be continuously introduced, but in order for a second detectably labeled nucleotide to incorporate into the extended sequencing primer, nucleotides in the first population of nucleotides that have not incorporated into a sequencing primer are generally removed (e.g., by washing), and a second population of detectably labeled nucleotides are introduced into the reaction. Then, a second detectably labeled nucleotide (e.g., A, T, C, or G nucleotide) is incorporated by the same or a different polymerase to extend the already extended sequencing primer in the 5′ to 3′ direction using a complementary nucleotide (a second nucleotide residue) in the template nucleotide as template. Thus, in some embodiments, cycles of introducing and removing detectably labeled nucleotides are performed.

In some embodiments, the base-by-base sequencing comprises using a polymerase that is fluorescently labeled and one or more nucleotides that are not fluorescently labeled. In some embodiments, the base-by-base sequencing comprises using a polymerase-nucleotide conjugate comprising a fluorescently labeled polymerase linked to a nucleotide moiety that is not fluorescently labeled. In some embodiments, the base-by-base sequencing comprises using a multivalent polymer-nucleotide conjugate comprising a polymer core, multiple nucleotide moieties, and one or more fluorescent labels.

In some embodiments, nucleic acid hybridization can be used for sequencing RCPs on the substrate. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004), all of which are herein incorporated by reference in their entireties.

In some embodiments, real-time monitoring of DNA polymerase activity can be used during sequencing of RCPs on the substrate. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181, all of which are herein incorporated by reference in their entireties.

### IV. Samples, Analytes, and Target Sequences

In some aspects, provided herein are methods for detecting one or more target nucleic acid sequences in a biological sample.

A. Samples

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can include nucleic acids (such as DNA or RNA). The biological sample can be obtained as a tissue sample, such as a tissue section, a cell block, a cell pellet, biopsy, a core biopsy, needle aspirate, or fine needle aspirate. The sample can be a skin sample, a colon sample, a histology sample, a histopathology sample, a tumor sample, living cells, cultured cells, or a clinical sample such as, for example, cultured tissues or cells, including cell suspensions. In some embodiments, the biological sample may comprise cells which are deposited on a surface.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Sample Permeabilization and Treatment

In some embodiments, a biological sample can be permeabilized to facilitate transfer of analytes or products thereof (such as circularized probes) out of the sample, and/or to facilitate transfer of species (such as probes) into the sample.

In some embodiments, a biological sample can be permeabilized to facilitate transfer of species (such as circularizable probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable. In some embodiments, after a circularized probe is hybridized to a target nucleic acid and circularized in a biological sample, the biological sample can be permeabilized and/or digested to facilitate transfer of the circularized probe from the sample towards immobilized oligonucleotides on a substrate, thereby facilitating capture of the circularized probe by the immobilized oligonucleotides.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### (ii) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

(iii) Freezing, Fixation, and Postfixation

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more circularizable probes or probe sets. In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a circularizable probe or probe set.

A post-fixing step may be performed using any suitable fixation reagent disclosed herein, for example, 3% (w/v) paraformaldehyde in DEPC-PBS.

### (iv) Embedding

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, the entire contents of which are incorporated herein by reference.

### (v) Crosslinking and De-Crosslinking

In some embodiments, the biological sample is reversibly cross-linked prior to or during an in situ assay module (e.g., imaging the biological sample prior to transfer of circularized probes to the substrate). In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible or irreversible crosslinking of the modified probe to the matrix.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

### (vi) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, e.g., to generate cDNA, thereby selectively enriching these RNAs.

In some aspects, when two or more analytes are analyzed, a first and second probe that is specific for (e.g., specifically hybridizes to) each RNA or cDNA analyte can be used. For example, in some embodiments of the methods provided herein, templated ligation is used to detect gene expression in a biological sample. An analyte of interest (such as a protein), bound by a labeling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include extension or amplification of templated ligation products (e.g., by rolling circle amplification of a circular product generated in a templated ligation reaction).

B. Analytes

The methods and compositions disclosed herein can be used to detect and analyze a wide variety of different analytes e.g., using the probes described in Section III. In some aspects, an analyte can include any biological substance, structure, moiety, or component to be analyzed. In some aspects, a target disclosed herein may similarly include any analyte of interest. In some examples, a target or analyte can be directly or indirectly detected.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe or probe set). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.).

### (i) Endogenous Analytes

In some embodiments, an analyte herein is endogenous to a biological sample and can include nucleic acid analytes and non-nucleic acid analytes. Methods and compositions disclosed herein can be used to analyze nucleic acid analytes (e.g., using a circularizable nucleic acid probe or probe set as described in Section III that directly or indirectly hybridizes to a nucleic acid analyte) and/or non-nucleic acid analytes (e.g., using a labeling agent that comprises a reporter oligonucleotide and binds directly or indirectly to a non-nucleic acid analyte) in any suitable combination.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

### (ii) Labeling Agents

In some embodiments, provided herein are methods and compositions for analyzing endogenous analytes (e.g., RNA, ssDNA, cell surface or intracellular proteins, and/or metabolites) in a sample using one or more labeling agents. In some embodiments, an analyte labeling agent may include an agent that interacts with an analyte (e.g., an endogenous analyte in a sample). In some embodiments, a sample can be stained, e.g., using a morphological stain (for instance, Hematoxylin & Eosin) or immunofluorescent staining, and be contacted with circularizable probes and the probes can be ligated in the sample, followed by capturing the circularized probes using primer sequences in oligonucleotide molecules immobilized on a substrate for on-substrate RCA (as described in Section II). In some embodiments, the labeling agents can comprise a reporter oligonucleotide that is indicative of the analyte or portion thereof interacting with the labeling agent. For example, the reporter oligonucleotide may comprise a barcode sequence that permits identification of the labeling agent. In some cases, the sample contacted by the labeling agent can be further contacted with a probe (e.g., a single-stranded probe sequence), that hybridizes to a reporter oligonucleotide of the labeling agent, in order to identify the analyte associated with the labeling agent. In some embodiments, the analyte labeling agent comprises an analyte binding moiety and a labeling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing (also referred to as post-fixation) steps after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of exemplary labeling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or epitope-binding fragments thereof. The antibodies or epitope-binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labeling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labeling agents are the same. In some embodiments in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some aspects, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using the optical detection techniques described herein.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some embodiments, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labeling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

C. Target Sequences

A target sequence for a probe disclosed herein (e.g., a circularizable probe or probe set as described in Section III) may be comprised in or associated with any analyte disclose herein, including an endogenous analyte (e.g., a cellular nucleic acid).

In some embodiments described herein, the analyte comprises or is associated with a target sequence. In some embodiments, a target sequence for a nucleic acid probe described herein is a marker sequence for a given analyte. A marker sequence is a sequence that identifies a given analyte (e.g., alone or in combination with one or more other marker sequences). Thus, in some embodiments, a marker sequence for a given target analyte is specific to that analyte, or unique, such that multiple target analytes can be distinguished from each other.

A “marker sequence” is thus a sequence which marks, is associated with, or identifies a given analyte. It is a sequence by which a given analyte may be detected and distinguished from other analytes. Where an “analyte” comprises a group of related molecules e.g. isoforms or variants or mutants etc., or molecules in a particular class or group, it is not required that a marker is unique or specific to only one particular analyte molecule, and it may be used to denote or identify the analyte as a group. However, where desired, a marker sequence may be unique or specific to a particular specific analyte molecule, e.g. a particular variant. In this way different variants, or isoforms, or mutants may be identified or distinguished from one another.

Where the analyte is a nucleic acid molecule, the target sequence (e.g., a marker sequence) may be a sequence present in the target analyte molecule, or a complement thereof (e.g. a reverse complement thereof). It may therefore be or comprise a variant or mutant sequence etc. present in the analyte, or a conserved sequence present in an analyte group which is specific to that group. The target sequence (e.g., a marker sequence) may alternatively be present in or incorporated into a product of an endogenous analyte as a tag or identifier (ID) sequence (e.g. a barcode) for the analyte or labeling agent. It may thus be a synthetic or artificial sequence.

In some aspects, one or more of the target sequences includes one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

### V. Compositions and Kits

Also provided herein are kits, for example comprising one or more polynucleotides disclosed herein, and reagents for performing the methods provided herein, for example reagents required for one or more steps comprising hybridization, ligation, amplification, detection, sequencing, immobilized oligonucleotide coated-substrate preparation, circularized probe capture, and/or sample preparation as described herein. In some embodiments, the kit comprises one or more substrates (e.g., a first substrate and/or a second substrate). In some embodiments, the one or more substrates are compatible for use with a sandwiching process facilitated by a device, sample holder, sample handling apparatus, and/or system described in, e.g., US. Patent Application Pub. No. 20210189475, US 2023/0017773 A1, and WO 2022/061152 A2, all of which are herein incorporated by reference in their entireties. In some embodiments, the kit comprises one or more reagents (e.g., permeabilization reagent) for the sandwiching process (e.g., described in Section II). In some examples, a substrate may comprise a plurality of capture agents (e.g., capture probes) directly or indirectly immobilized thereon. In some embodiments, the kit further comprises a target nucleic acid. In some embodiments, any or all of the polynucleotides are DNA molecules. In some embodiments, the target nucleic acid is a messenger RNA molecule. In some embodiments, the kit comprises one or more reagents for migration of circularized probes (e.g., the reagent medium) as described in Section III. In some embodiments, the kit comprises one or more reagents for staining the biological sample. In some embodiments, the kit comprises a morphological stain (e.g., Hematoxylin & Eosin) or an immunofluorescent staining.

The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits contain reagents for detection and/or sequencing, such as barcode detection probes or detectable labels. In some embodiments, the kits optionally contain other components, for example nucleic acid primers, enzymes and reagents, buffers, nucleotides, modified nucleotides, reagents for additional assays.

In some embodiments, provided herein are systems for performing the methods described herein including support devices, sample holders, sample handling apparatuses, or systems for implementing a sandwiching process are described in, e.g., US. Patent Application Pub. No. 20210189475, and PCT/US2021/050931, all of which are herein incorporated by reference in their entireties.

### VI. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

“Hybridization” as used herein may refer to the process in which two single-stranded polynucleotides bind non-covalently to form a stable double-stranded polynucleotide. In one aspect, the resulting double-stranded polynucleotide can be a “hybrid” or “duplex.”

A “hybridization complex” as used herein may comprise one, two, or more strands or separate molecules. A hybridization complex that comprises three or more strands or separate molecules does not necessarily comprise direct hybridization between every possible pairwise combination thereof, so long as at least two molecules or strands are directly hybridized to each other, or are in the process of binding to or unbinding from each other, at a given time.

“Hybridization conditions” typically include salt concentrations of approximately less than 1 M, often less than about 500 mM and may be less than about 200 mM. A “hybridization buffer” includes a buffered salt solution such as 5% SSPE. Hybridization temperatures can be as low as 5° C., but are typically greater than 22° C., and more typically greater than about 30° C., and typically in excess of 37° C. Hybridizations are often performed under stringent conditions, e.g., conditions under which a sequence will hybridize to its target sequence but will not hybridize to other, non-complementary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C. lower than the T for the specific sequence at a defined ionic strength and pH. The melting temperature Tₘ can be the temperature at which a population of double-stranded nucleic acid molecules becomes half dissociated into single strands. Several equations for calculating the Tₘ of nucleic acids may be suitable. As indicated by standard references, a simple estimate of the Tₘ value may be calculated by the equation, Tₘ=81.5+0.41 (% G+C), when a nucleic acid is in aqueous solution at 1 M NaCl (see e.g., Anderson and Young, Quantitative Filter Hybridization, in Nucleic Acid Hybridization (1985)). Other references (e.g., Allawi and SantaLucia, Jr., Biochemistry, 36:10581-94 (1997)) include alternative methods of computation which take structural and environmental, as well as sequence characteristics into account for the calculation of Tₘ.

In general, the stability of a hybrid is a function of the ion concentration and temperature. Typically, a hybridization reaction is performed under conditions of lower stringency, followed by washes of varying, but higher, stringency. Exemplary stringent conditions include a salt concentration of at least 0.01 M to no more than 1 M sodium ion concentration (or other salt) at a pH of about 7.0 to about 8.3 and a temperature of at least 25° C. For example, conditions of 5×SSPE (750 mM NaCl, 50 mM sodium phosphate, 5 mM EDTA at pH 7.4) and a temperature of approximately 30° C. are suitable for allele-specific hybridizations, though a suitable temperature depends on the length and/or GC content of the region hybridized. In one aspect, “stringency of hybridization” in determining percentage mismatch can be as follows: 1) high stringency: 0.1×SSPE, 0.1% SDS, 65° C.; 2) medium stringency: 0.2×SSPE, 0.1% SDS, 50° C. (also referred to as moderate stringency); and 3) low stringency: 1.0×SSPE, 0.1% SDS, 50° C. It is understood that equivalent stringencies may be achieved using alternative buffers, salts and temperatures. For example, moderately stringent hybridization can refer to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions can be conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5×Denhardt's solution, 5×SSPE, 0.2% SDS at 42° C., followed by washing in 0.1×SSPE, and 0.1% SDS at 65° C. Low stringency hybridization can refer to conditions equivalent to hybridization in 10% formamide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M EDTA. Other suitable moderate stringency and high stringency hybridization buffers and conditions are described, for example, in Sambrook et al., Molecular Cloning: A Laboratory Manual, 2nd ed., Cold Spring Harbor Press, Plainview, N.Y. (1989); and Ausubel et al., Short Protocols in Molecular Biology, 4th ed., John Wiley & Sons (1999).

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See M. Kanehisa, Nucleic Acids Res. 12:203 (1984).

A “primer” used herein can be an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

“Ligation” may refer to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide.

“Sequencing,” “sequence determination” and the like means determination of information relating to the nucleotide base sequence of a nucleic acid. Such information may include the identification or determination of partial as well as full sequence information of the nucleic acid. Sequence information may be determined with varying degrees of statistical reliability or confidence. In one aspect, the term includes the determination of the identity and ordering of a plurality of contiguous nucleotides in a nucleic acid. “High throughput digital sequencing” or “next generation sequencing” means sequence determination using methods that determine many (typically thousands to billions) of nucleic acid sequences in an intrinsically parallel manner, e.g. where DNA templates are prepared for sequencing not one at a time, but in a bulk process, and where many sequences are read out preferably in parallel, or alternatively using an ultra-high throughput serial process that itself may be parallelized. Such methods include but are not limited to pyrosequencing (for example, as commercialized by 454 Life Sciences, Inc., Branford, Conn.); sequencing by ligation (for example, as commercialized in the SOLiD™ technology, Life Technologies, Inc., Carlsbad, Calif); sequencing by synthesis using modified nucleotides (such as commercialized in TruSeq™ and HiSeq™ technology by Illumina, Inc., San Diego, Calif.; HeliScope™ by Helicos Biosciences Corporation, Cambridge, Ma.; and PacBio RS by Pacific Biosciences of California, Inc., Menlo Park, Calif), sequencing by ion detection technologies (such as Ion Torrent™ technology, Life Technologies, Carlsbad, Calif); sequencing of DNA nanoballs (Complete Genomics, Inc., Mountain View, Calif.); nanopore-based sequencing technologies (for example, as developed by Oxford Nanopore Technologies, LTD, Oxford, UK), and like highly parallelized sequencing methods.

“SNP” or “single nucleotide polymorphism” may include a genetic variation between individuals; e.g., a single nitrogenous base position in the DNA of organisms that is variable. SNPs are found across the genome; much of the genetic variation between individuals is due to variation at SNP loci, and often this genetic variation results in phenotypic variation between individuals. SNPs for use in the present disclosure and their respective alleles may be derived from any number of sources, such as public databases (U.C. Santa Cruz Human Genome Browser Gateway (genome.ucsc.edu/cgi-bin/hgGateway) or the NCBI db SNP web site (www.ncbi.nlm.nih gov/SNP/), or may be experimentally determined as described in U.S. Pat. No. 6,969,589; and US Pub. No. 2006/0188875 entitled “Human Genomic Polymorphisms.” Although the use of SNPs is described in some of the embodiments presented herein, it will be understood that other biallelic or multi-allelic genetic markers may also be used. A biallelic genetic marker is one that has two polymorphic forms, or alleles. As mentioned above, for a biallelic genetic marker that is associated with a trait, the allele that is more abundant in the genetic composition of a case group as compared to a control group is termed the “associated allele,” and the other allele may be referred to as the “unassociated allele.” Thus, for each biallelic polymorphism that is associated with a given trait (e.g., a disease or drug response), there is a corresponding associated allele. Other biallelic polymorphisms that may be used with the methods presented herein include, but are not limited to multinucleotide changes, insertions, deletions, and translocations. It will be further appreciated that references to DNA herein may include genomic DNA, mitochondrial DNA, episomal DNA, and/or derivatives of DNA such as amplicons, RNA transcripts, cDNA, DNA analogs, etc. The polymorphic loci that are screened in an association study may be in a diploid or a haploid state and, ideally, would be from sites across the genome.

“Multiplexing” or “multiplex assay” herein may refer to an assay or other analytical method in which the presence and/or amount of multiple targets, e.g., multiple nucleic acid target sequences, can be assayed simultaneously by using more than one capture probe conjugate, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

The term “adjacent” as used herein includes but is not limited to being directly linked by a phosphodiester bond. For example, “adjacent” nucleotides or regions on a nucleic acid such as a probe may be separated by a number of nucleotides. For instance, a toehold region and an interrogatory region adjacent to each other may be separated by 0, 1, 2, 3, 4, 5, 6, 7, 8, 9, 10 or more nucleotides in a probe.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

The term “each,” when used in reference to a collection of items, is intended to identify an individual item in the collection but does not necessarily refer to every item in the collection, unless expressly stated otherwise, or unless the context of the usage clearly indicates otherwise.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

The following examples are included for illustrative purposes only and are not intended to limit the scope of the present disclosure.

### Example 1: On-Substrate Extension of Immobilized Oligonucleotides Using Various Lengths of Immobilized Capture Oligonucleotide Molecules

This example demonstrates that including a flexible linker between the capture region comprising the primer sequence used for priming and the immobilized 5′ end of the oligonucleotide molecules improves the ability to perform on-substrate primer extension using a polymerase.

A substrate comprising a lawn of immobilized oligonucleotides was prepared by dipping a glass slide with a high density amine-binding/NHS ester coating (Surmodics™ TRIDIA™ HD Activated Slides) in a solution comprising 5′ amine modified oligonucleotide molecules (10 μM oligonucleotide molecules in DNA printing buffer). The 5′ amine was reacted with the NHS ester to immobilize the oligonucleotides in a lawn covering the capture area of the substrate.

In this example, the capture region of the immobilized oligonucleotide molecules was the reverse complement of an 18s rRNA sequence to enable capture and cDNA synthesis from 18s rRNA. Immobilized oligonucleotide molecules of different lengths (25 nucleotides or 75 nucleotides, each oligonucleotide molecule comprising a capture region of the same length) were tested to determine the effect of adding a flexible linker (50 nucleotides long) between the capture region and the immobilized 5′ end.

Paraffin embedded formalin fixed (FFPE) mouse brain tissue samples were cryosectioned onto the oligonucleotide coated slides. The tissue was permeabilized and 18s rRNA was migrated towards the substrate by diffusion, after which the tissue was removed from the substrate with captured 18s rRNA.

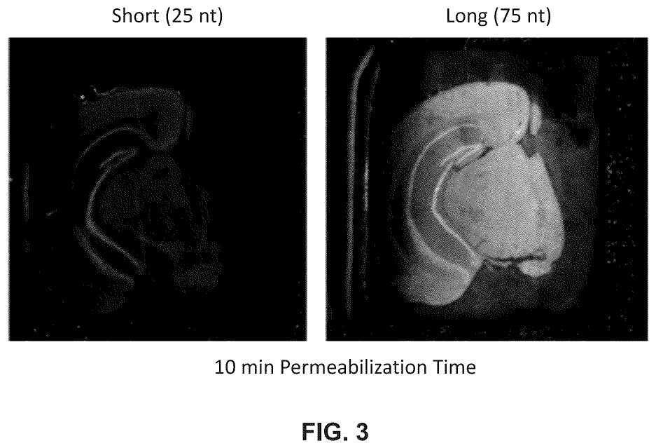

After primer extension of the capture region using a polymerase and the captured 18s rRNA as template, the extended immobilized oligonucleotides were detected. As shown in FIG. 3, the longer (75nt) oligonucleotide molecules on the slides yielded higher intensity of signals, indicating that a flexible linker between the substrate and a capture region used as a primer provides greater primer extension efficiency. These results suggest that a flexible linker between an immobilized end of an oligonucleotide molecule and a capture region used as a primer can provide high efficiency of priming for on-substrate rolling circle amplification.

### Example 2: Analysis by On-Substrate RCA

This example demonstrates that analysis of a target nucleic acid sequence in a biological sample using on-substrate rolling circle amplification provided high quality, well-resolved RCPs and analysis of the positions of analytes in a biological sample.

### Substrate Preparation

In this example, the biological samples (tissue samples) were on a first substrate. Circularizable probes were ligated to form circularized probes upon hybridization to target nucleic acid sequences in the biological samples. The first substrate (e.g., slide) was aligned with a second substrate (a glass slide coated with immobilized oligonucleotide molecules) on a transfer instrument so that the tissue sample aligned with a “capture area” comprising the lawn of immobilized oligonucleotide molecules. The tissue sample was permeabilized (e.g., digested) and the circularized probes were migrated to the second substrate for capture and rolling circle amplification of the circularized probes.

To prepare the biological samples on the first substrate, fresh frozen or paraffin embedded formalin fixed (FFPE) mouse brain tissue samples were cryosectioned onto SuperFrost® Plus slides (glass slides) for processing. For fresh frozen samples, the tissue sections were treated with formaldehyde, and washed in sodium dodecyl sulfate (SDS) and methanol. FFPE samples were de-crosslinked in citrate buffer.

The second substrate comprising a lawn of immobilized oligonucleotides was prepared by dipping a glass slide with a high density amine-binding/NHS ester coating (Surmodics™ TRIDIA™ HD Activated Slides) in a solution comprising 5′ amine modified oligonucleotide molecules (10 μM oligonucleotide molecules in DNA printing buffer). The 5′ amine was reacted with the NHS ester to immobilize the oligonucleotides in a lawn covering the capture area of the second substrate. The immobilized oligonucleotide molecules had a capture region at the free 3′ end. The capture region was designed to be complementary to a primer binding region in the circularized probes, allowing the capture region to “capture” circularized probes by hybridization, and to be extended by a polymerase to form a rolling circle amplification product using the captured circularized probe as a template.

### Circularized Probe Hybridization and Ligation

To prepare for probe hybridization, a wash buffer was added to the tissue sections. Sets of circularizable probes (each with a common sequence complementary to the capture region of the immobilized oligonucleotide molecules on the substrate) were used to target different genes such that different cell types in the brain samples can be distinguished, in addition to negative control. For example, gene expression in excitatory, inhibitory neurons and dentate gyrus cells were detected with sets of circularizable probes. In addition to a target hybridization region designed such that hybridization to the target nucleic acid sequence brings a ligatable 5′ end and 3′ end of the circularizable probe into proximity for ligation, each circularizable probe also contained a common primer binding region (complementary to the primer sequence of the capture region of the immobilized oligonucleotide molecules on the second substrate) and a barcode region. The probes were incubated with the tissue sections and hybridization buffer including SSC and formamide for hybridization of the probe sets to the target nucleic acid sequences in the sample. After probe hybridization, the probe hybridization mixture was removed and the samples were washed to remove unbound probes.

The circularizable probes were then ligated to generate circularized probes, and the tissue sections were then washed in PBST or 2×SSC. The tissue sections were then permeabilized (e.g., digested) using a RNase H/Proteinase K mixture to release the circularized probes.

### Circularized Probe Capture and RCA

The circularized probes were then transferred to the second substrate comprising the immobilized oligonucleotide molecules by passive diffusion (10 minute (10′), 30 minute (30′) and 60 minute (60′) incubations at 37° C. were tested) using a transfer instrument to perform the sandwiching process as described herein. Circularized probes were captured by hybridization to the capture region of the immobilized oligonucleotide molecules, which was also used as the primer region for RCA.

The slides were incubated with a RCA mixture (containing a Phi29 DNA polymerase and dNTPs for RCA of the circular probes) at 30° C. for 1.5 hours. The slides were then washed in TE pH 8.0 buffer three times. Hybridization probes in a hybridization buffer (e.g., containing SSC and formamide) were hybridized to RCA products (RCPs) on the slides. The hybridization probes included probes that hybridize to the barcode sequence in the RCPs (which is complementary to the barcode sequence in the circularized probes) and comprise overhangs for hybridization of fluorescently labelled detection oligonucleotides.

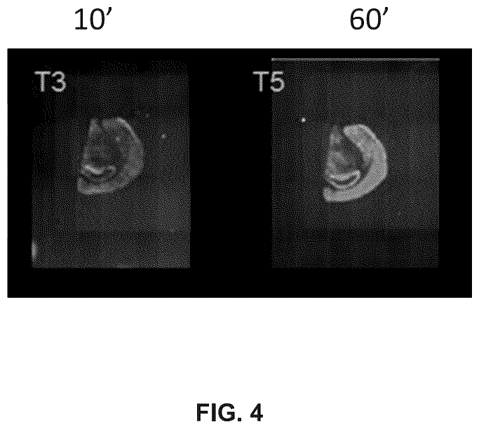

As shown in FIG. 4, transferring the circularized probes for 60 minutes yielded the most signal, indicating more circularized probes were captured with a longer transfer time compared to 10 minutes.

To test whether using a crowding agent can affect the quality of the RCPs, a similar experiment substantially as described above was performed and RCAs were carried out with or without 10% PEG20k 1\4W in the RCA mixture. The slides were imaged in fluorescent microscope with 20× or 40× objective.

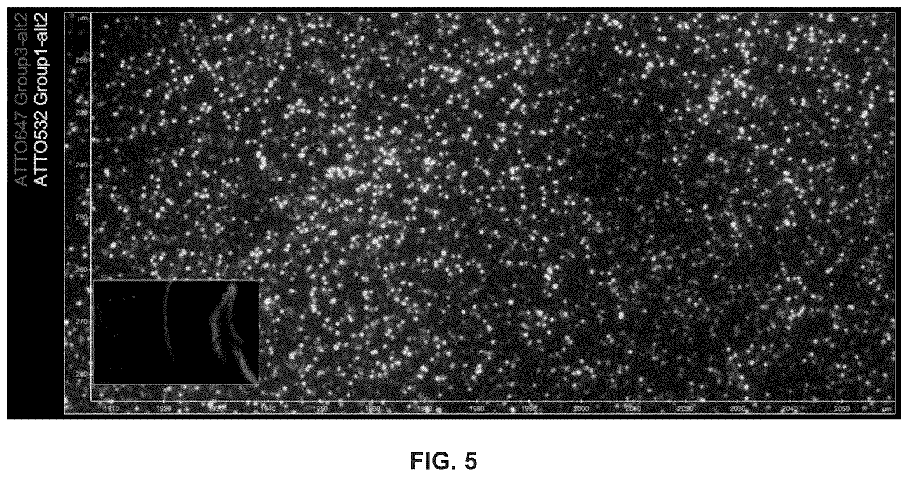

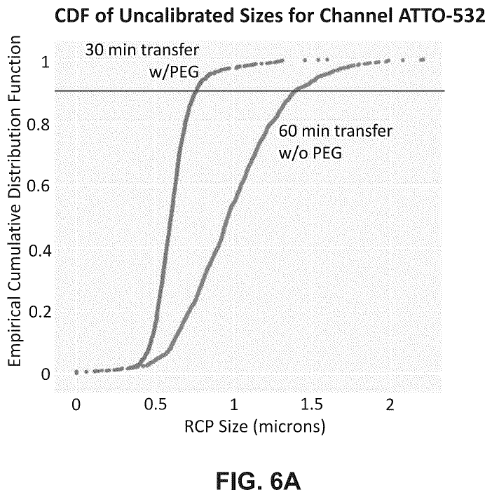

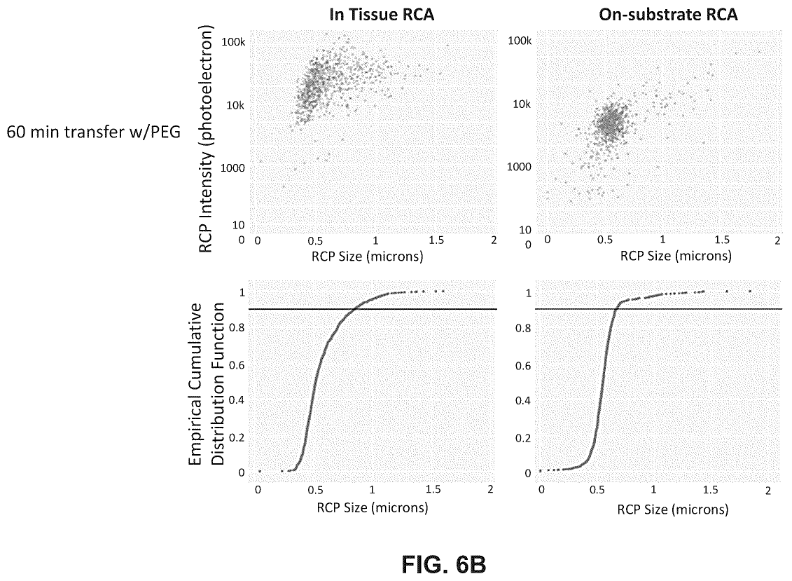

The results show that the addition of a crowding agent such as PEG helps with the generation of more uniform RCPs, even compared to longer transfer times without PEG (FIGS. 5 and 6A). Compared to amplification products generated in a tissue sample, RCPs generated using circularized probes transferred and captured on the substrate was more compact and uniform in size and signal intensity (FIG. 6B). In some aspects, the methods provided herein including circularized probe capture and amplification on substrate can be advantageous over tissue based methods.

### Example 3: On-Substrate RCA of Circularized Probes from Samples that have been Stained

This example demonstrates that samples that have been stained, e.g., using a morphological stain (for instance, Hematoxylin & Eosin) or immunofluorescent staining, can be contacted with circularizable probes and the probes can be ligated in the sample, followed by capturing the circularized probes using primer sequences in oligonucleotide molecules immobilized on a substrate for on-substrate RCA.

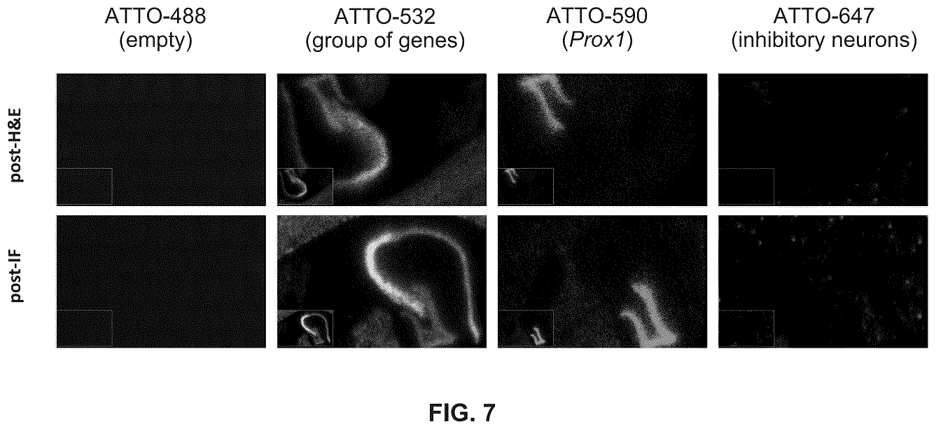

FFPE mouse brain tissue samples were subjected to immunofluorescent staining (IF) using DAPI or H&E staining. Immunofluorescent staining and morphological stains can be used to illustrate cell boundaries and cellular structures. For instance, DAPI is used to visualize nuclear DNA, and H&E staining is used to visualize cell nuclei and the extracellular matrix and cytoplasm. The post-H&E and the post-IF samples were contacted with circularizable probes followed by probe ligation in the samples and capturing the circularized probes by oligonucleotide molecules on substrates, essentially as described in Example 2. After on-substrate RCA using the immobilized oligonucleotide molecules as primers and the captured circularized probes as templates, the RCA products were detected using intermediate probes that hybridize to the RCA products and fluorescently labeled probes that in turn hybridize to the intermediate probes. As shown in FIG. 7, target nucleic acid analyses using the methods disclosed herein showed sensitive detection of RCA products generated on substrates for the DAPI-stained samples (“post-IF”) and the H&E stained samples (“post-H&E”). The use of DAPI or H&E can help correlate the locations of the detected RCPs on substrates with locations of cells in the biological sample.

The present disclosure is not intended to be limited in scope to the particular disclosed embodiments, which are provided, for example, to illustrate various aspects of the disclosure. Various modifications to the compositions and methods described will become apparent from the description and teachings herein. Such variations may be practiced without departing from the true scope and spirit of the disclosure and are intended to fall within the scope of the present disclosure.

1-67. (canceled)

68. A method of analyzing a biological sample, comprising:
a) contacting the biological sample with a circularizable probe or probe set that binds to a target nucleic acid sequence in the biological sample;
b) ligating the circularizable probe or probe set to generate a circularized probe at a location in the biological sample, wherein the circularized probe comprises a primer binding sequence;
c) transferring the circularized probe from the biological sample to a substrate comprising a plurality of oligonucleotide molecules immobilized on the substrate, wherein at least a portion of the immobilized oligonucleotide molecules comprise a primer sequence, and wherein the primer sequence is bound to the primer binding sequence of the circularized probe;
d) using a polymerase and the primer sequence to perform rolling circle amplification of the circularized probe, thereby generating a rolling circle amplification product immobilized at a location on the substrate; and
e) detecting the rolling circle amplification product at the location on the substrate.

69. The method of claim 68, wherein contacting the biological sample with the circularizable probe or probe set in a) comprises contacting the biological sample with a pool of circularizable probes or probe sets that bind to a plurality of different target nucleic acid sequences in the biological sample, wherein the pool comprises the circularizable probe or probe set, and wherein:
the ligating in b) comprises ligating at least a subset of the plurality of circularizable probes or probe sets to generate a plurality of circularized probes at two or more locations in the biological sample;
the transferring in c) comprises transferring at least a subset of the circularized probes from the biological sample to the substrate;
performing rolling circle amplification in d) comprises performing rolling circle amplification of at least a subset of the circularized probes using the polymerase and the primer sequence, thereby generating a plurality of rolling circle amplification products immobilized at two or more locations on the substrate; and
the detecting in e) comprises detecting at least a subset of the rolling circle amplification products at the two or more locations on the substrate.

70. The method of claim 68, wherein the biological sample is on a first substrate in the contacting in a), and the substrate comprising the plurality of oligonucleotide molecules immobilized on the substrate is a second substrate.

71. The method of claim 70, wherein the biological sample does not come into direct contact with the second substrate.

72. The method of claim 68, wherein the immobilized oligonucleotide molecules are uniformly distributed on a surface of the substrate.

73. The method of claim 68, wherein the immobilized oligonucleotide molecules are distributed over a capture area on the substrate, wherein the capture area corresponds to an area aligned with the biological sample.

74. The method of claim 73, wherein the immobilized oligonucleotide molecules are not distributed in a pattern of discrete features over the capture area.

75. The method of claim 68, wherein the primer sequence is a common sequence among at least two of the immobilized oligonucleotide molecules.

76. The method of claim 68, wherein the primer sequence is at the 3′ end of the immobilized oligonucleotide molecules.

77. The method of claim 68, wherein the primer sequence is connected to the substrate via a flexible linker.

78. The method of claim 77, wherein the flexible linker is a nucleic acid sequence of between about 5 and about 100 nucleotides in length between the primer sequence and an immobilized end of the immobilized oligonucleotide molecules.

79. The method of claim 78, wherein the flexible linker is at least 75 nucleotides in length.

80. The method of claim 68, wherein the biological sample is a tissue section.

81. The method of claim 68, wherein the target nucleic acid sequence is RNA.

82. The method of claim 68, wherein the transferring in c) comprises at least 60 minutes of passive diffusion.

83. The method of claim 68, wherein the rolling circle amplification is performed in a buffer comprising a crowding agent selected from the group consisting of poly(ethylene glycol) (PEG), glycerol, Ficoll, and dextran sulfate.

84. The method of claim 83, wherein the crowding agent is poly(ethylene glycol) (PEG).

85. The method of claim 68, wherein the biological sample has been stained with hematoxylin and eosin (H&E).

86. The method of claim 68, comprising imaging the biological sample and correlating the location or locations of the rolling circle amplification product(s) on the substrate with a location or locations in an image of the biological sample.

87. The method of claim 54, wherein the detecting in e) comprises detecting a detectably labeled probe or probes bound directly or indirectly to the rolling circle amplification product or products.
